(19) United States
(12) Patent Application Publication
(10) Pub. No.: US 2006/0100230 A1
(43) Pub. Date: May 11, 2006

(54) Indolin phenylsulfonamide derivatives

(76) Inventors: Hilmar Bischoff, Wuppertal (DE); Elke Dittrich-Wengenroth, Wuppertal (DE); Martina Wuttke, Wuppertal (DE); Heike Heckroth, Wuppertal (DE); Wolfgang Thielemann, Wuppertal (DE); Michael Woltering, Wuppertal (DE); Michael Otteneder, Arlesheim (CH)

Correspondence Address: JEFFREY M. GREENMAN, BAYER PHARMACEUTICALS CORPORATION, 400 MORGAN LANE, WEST HAVEN, CT, 06516, US

(21) Appl. No.: 10/519,125

(22) PCT Filed: Jun. 30, 2003
(86) PCT No.: PCT/EP03/06896

§ 371 Date: Aug. 8, 2005

(30) Foreign Application Priority Data
Jul. 3, 2002 (SE) 10229777.0

(51) Int. Cl. A61K 31/4745; A61K 31/404; C07D 471/02; C07D 405/02

(52) U.S. Cl. 514/300; 514/418; 514/414; 546/113; 548/454

(57) ABSTRACT

The invention relates to novel substituted indolin phenylsulfonamide derivatives, to a method for the production thereof and to the use thereof in medicaments, especially as potent PPAR-delta activating compounds for the prophylaxis and/or treatment of cardiovascular diseases, especially dyslipidaemia and coronary heart diseases.

The present application relates to novel substituted indolinephenylsulphonamide derivatives, to processes for their preparation and to their use in medicaments, in particular as potent PPAR-delta-activating compounds for the prophylaxis and/or treatment of cardiovascular disorders, in particular dyslipidaemias, arteriosclerosis and coronary heart diseases.

In spite of many successful therapies, coronary heart diseases (CHDs) remain a serious public health problem. Treatment with statins, which inhibit HMG-CoA reductase, very successfully lowers the LDL cholesterol plasma concentration, resulting in a significant reduction of the mortality of patients at risk; however, convincing treatment strategies for the therapy of patients having an unfavourable HDL/LDL cholesterol ratio and/or hypertriglyeridaemia are still not available to date.

Currently, fibrates are the only therapy option for patients of these risk groups. They act as weak agonists of the peroxisome-proliferator-activated receptor (PPAR)-alpha (Nature 1990, 347, 645-50). A disadvantage of fibrates which have hitherto been approved is that their interaction with the receptor is only weak, requiring high daily doses and causing considerable side-effects.

For the peroxisome-proliferator-activated receptor (PPAR)-delta (Mol. Endocrinol. 1992, 6, 1634-41), first pharmacological findings in animal models indicate that potent PPAR-delta-agonists may likewise lead to an improvement in the HDL/LDL cholesterol ratio and in hypertriglyceridaemia.

WO 00/23407 discloses PPAR modulators for treating obesity, atherosclerosis and/or diabetes. WO 93/15051 and EP 636 608-A1 describe 1-benzenesulphonyl-1,3-dihydroindol-2-one derivatives as vasopressin and/or oxytocin antagonists for the treatment of various disorders.

It was an object of the present invention to provide novel compounds suitable for use as PPAR-delta modulators.

It has now been found that compounds of the general formula (I) 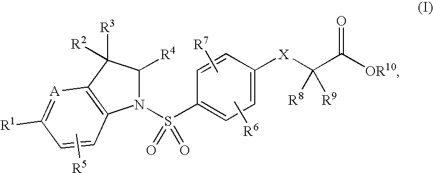
in which
A represents the group C—R¹¹ or represents N,
   where
   R¹¹ represents hydrogen or (C₁-C₄)-alkyl,
X represents O, S or CH₂,
R₁ represents (C₆-C₁₀)-aryl or represents 5- to 10-membered heteroaryl having up to three heteroatoms from the group consisting of N, O and S, which radicals may for their part each be mono- to trisubstituted by identical or different substituents selected from the group consisting of halogen, cyano, nitro, (C₁-C₆)-alkyl (which for its part may be substituted by hydroxyl), (C₁-C₆)-alkoxy, phenoxy, benzyloxy, trifluoromethyl, trifluoromethoxy, (C₂-C₆)-alkenyl, phenyl, benzyl, (C₁-C₆)-alkylthio, (C₁-C₆)-alkylsulphonyl, (C₁-C₆)-alkanoyl, (C₁-C₆)-alkoxycarbonyl, carboxyl, amino, (C₁-C₆)-acylamino, mono- and di-(C₁-C₆)-alkylamino and 5- or 6-membered heterocyclyl having up to two heteroatoms from the group consisting of N, O and S,
   or represents a group of the formula 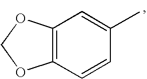
R² and R³ are identical or different and independently of one another represent hydrogen or (C₁-C₆)-alkyl or together with the carbon atom to which they are attached form a 3- to 7-membered spiro-linked cycloalkyl ring,
R⁴ represents hydrogen or (C₁-C₆)-alkyl,
R⁵ represents hydrogen or (C₁-C₆)-alkyl,
R⁶ represents hydrogen or (C₁-C₆)-alkyl,
R⁷ represents hydrogen, (C₁-C₆)-alkyl, (C₁-C₆)-alkoxy or halogen,
R⁸ and R⁹ are identical or different and independently of one another represent hydrogen or (C₁-C₄)-alkyl, and
R¹⁰ represents hydrogen or represents a hydrolysable group which can be degraded to the corresponding carboxylic acid,
and their pharmaceutically acceptable salts, solvates and solvates of the salts,
have pharmacological action and can be used as medicaments or for preparing medicament formulations.

In the context of the invention, in the definition of R¹⁰, a hydrolysable group means a group which, in particular in the body, causes the —C(O)OR¹⁰ grouping to be converted into the corresponding carboxylic acid (R¹⁰=hydrogen). Such groups are, by way of example and by way of preference: benzyl, (C₁-C₆)-alkyl or (C₃-C₈)-cycloalkyl which are in each case optionally mono- or polysubstituted by identical or different substituents from the group consisting of halogen, hydroxyl, amino, (C₁-C₆)-alkoxy, carboxyl, (C₁-C₆)-alkoxycarbonyl, (C₁-C₆)-alkoxycarbonylamino or (C₁-C₆)-alkanoyloxy, or in particular (C₁-C₄)-alkyl which is optionally mono- or polysubstituted by identical or different substituents from the group consisting of halogen, hydroxyl, amino, (C₁-C₄)-alkoxy, carboxyl, (C₁-C₄)-alkoxycarbonyl, (C₁-C₄)-alkoxycarbonylamino or (C₁-C₄)-alkanoyloxy.

In the context of the invention, (C₁-C₆)-alkyl and (C₁-C₄)-alkyl represent a straight-chain or branched alkyl radical having 1 to 6 and 1 to 4 carbon atoms, respectively. Preference is given to a straight-chain or branched alkyl radical having 1 to 4 carbon atoms. The following radicals may be mentioned by way of example and by way of preference: methyl, ethyl, n-propyl, isopropyl and t-butyl.

In the context of the invention, (C₂-C₆)-alkenyl represents a straight-chain or branched alkenyl radical having 2 to 6 carbon atoms. Preference is given to a straight-chain or branched alkenyl radical having 2 to 4 carbon atoms. The following radicals may be mentioned by way of example and by way of preference: vinyl, allyl, isopropenyl and n-but-2-en-1-yl.

In the context of the invention, (C₃-C₈)-cycloalkyl represents a monocyclic cycloalkyl group having 3 to 8 carbon atoms. The following radicals may be mentioned by way of example and by way of preference: cyclopropyl, cyclobutyl, cyclopentyl and cyclohexyl.

In the context of the invention, (C₆-C₁₀)-aryl represents an aromatic radical having preferably 6 to 10 carbon atoms. Preferred aryl radicals are phenyl and naphthyl.

In the context of the invention, (C₁-C₆)-alkoxy and (C₁-C₄)-alkoxy represent a straight-chain or branched alkoxy radical having 1 to 6 and 1 to 4 carbon atoms, respectively. Preference is given to a straight-chain or branched alkoxy radical having 1 to 4 carbon atoms. The following radicals may be mentioned by way of example and by way of preference: methoxy, ethoxy, n-propoxy, isopropoxy and t-butoxy.

In the context of the invention, (C₁-C₆)-alkoxycarbonyl and (C₁-C₄)-alkoxycarbonyl represent a straight-chain or branched alkoxy radical having 1 to 6 and 1 to 4 carbon atoms, respectively, which radical is attached via a carbonyl group. Preference is given to a straight-chain or branched alkoxycarbonyl radical having 1 to 4 carbon atoms. The following radicals may be mentioned by way of example and by way of preference: methoxycarbonyl, ethoxycarbonyl, n-propoxycarbonyl, isopropoxycarbonyl and t-butoxycarbonyl.

In the context of the invention, (C₁-C₆)-alkoxycarbonylamino and (C₁-C₄)-alkoxycarbonylamino represent an amino group having a straight-chain or branched alkoxycarbonyl substituent which has 1 to 6 and 1 to 4 carbon atoms, respectively, in the alkoxy radical and which is attached via the carbonyl group. Preference is given to an alkoxycarbonylamino radical having 1 to 4 carbon atoms. The following radicals may be mentioned by way of example and by way of preference: methoxycarbonylamino, ethoxycarbonylamino, n-propoxycarbonylamino and t-butoxycarbonylamino.

In the context of the invention, (C₁-C₆)-alkanoyl represents a straight-chain or branched alkyl radical having 1 to 6 carbon atoms which carries a doubly attached oxygen atom in the 1-position and is attached via the 1-position. Preference is given to a straight-chain or branched alkanoyl radical having 1 to 4 carbon atoms. The following radicals may be mentioned by way of example and by way of preference: formyl, acetyl, propionyl, n-butyryl, i-butyryl, pivaloyl and n-hexanoyl.

In the context of the invention, (C₁-C₆)-alkanoyloxy and (C₁-C₄)-alkanoyloxy represent a straight-chain or branched alkyl radical having 1 to 6 and 1 to 4 carbon atoms, respectively, which carries a doubly attached oxygen atom in the 1-position and is attached in the 1-position via a further oxygen atom. Preference is given to an alkanoyloxy radical having 1 to 4 carbon atoms. The following radicals may be mentioned by way of example and by way of preference: acetoxy, propionoxy, n-butyroxy, i-butyroxy, pivaloyloxy, n-hexanoyloxy.

In the context of the invention, mono-(C₁-C₆)-alkylamino and mono-(C₁-C₄)-alkylamino represent an amino group having a straight-chain or branched alkyl substituent of 1 to 6 and 1 to 4 carbon atoms, respectively. Preference is given to a straight-chain or branched monoalkylamino radical having 1 to 4 carbon atoms. The following radicals may be mentioned by way of example and by way of preference: methylamino, ethylamino, n-propylamino, isopropylamino and t-butylamino.

In the context of the invention, di-(C₁-C₆)-alkylamino and di-(C₁-C₄)-alkylamino represent an amino group having two identical or different straight-chain or branched alkyl substituents having in each case 1 to 6 and 1 to 4 carbon atoms, respectively. Preference is given to straight-chain or branched dialkylamino radicals having in each case 1 to 4 carbon atoms. The following radicals may be mentioned by way of example and by way of preference: N,N-dimethylamino, N,N-diethylamino, N-ethyl-N-methylamino, N-methyl-N-n-propylamino, N-isopropyl-N-n-propylamino, N-t-butyl-N-methylamino, N-ethyl-N-n-pentylamino and N-n-hexyl-N-methylamino.

In the context of the invention, (C₁-C₆)-acylamino represents an amino group having a straight-chain or branched alkanoyl substituent which has 1 to 6 carbon atoms and is attached via the carbonyl group. Preference is given to an acylamino radical having 1 or 2 carbon atoms. The following radicals may be mentioned by way of example and by way of preference: formamido, acetamido, propionamido, n-butyramido and pivaloylamido.

In the context of the invention, (C₁-C₆)-alkylthio represents a straight-chain or branched alkylthio radical having 1 to 6 carbon atoms. Preference is given to a straight-chain or branched alkylthio radical having 1 to 4 carbon atoms. The following radicals may be mentioned by way of example and by way of preference: methylthio, ethylthio, n-propylthio, isopropylthio, t-butylthio, n-pentylthio and n-hexylthio.

In the context of the invention, (C₁-C₆)-alkylsulphonyl represents a straight-chain or branched alkylsulphonyl radical having 1 to 6 carbon atoms. Preference is given to a straight-chain or branched alkylsulphonyl radical having 1 to 4 carbon atoms. The following radicals may be mentioned by way of example and by way of preference: methylsulphonyl, ethylsulphonyl, n-propylsulphonyl, isopropylsulphonyl, t-butylsulphonyl, n-pentylsulphonyl and n-hexylsulphonyl.

In the context of the invention, 5- to 10-membered and 5- or 6-membered heteroaryl having up to 3 or up to 2 identical or different heteroatoms, respectively, from the group consisting of N, O and S represents a mono- or optionally bicyclic aromatic heterocycle (heteroaromatic) which is attached via a ring carbon atom or, if appropriate, via a ring nitrogen atom of the heteroaromatic. Examples which may be mentioned are: furanyl, pyrrolyl, thienyl, pyrazolyl, imidazolyl, thiazolyl, oxazolyl, isoxazolyl, isothiazolyl, pyridyl, pyrimidinyl, pyridazinyl, pyrazinyl, benzofuranyl, benzothienyl, benzimidazolyl, benzoxazolyl, indolyl, indazolyl, quinolinyl, isoquinolinyl, naphthyridinyl, quinazolinyl, quinoxalinyl. Preference is given to 5- or 6-membered heteroaryl radicals having up to two nitrogen atoms, such as, for example, imidazolyl, pyridyl, pyrimidinyl, pyridazinyl, pyrazinyl.

In the context of the invention, 5- or 6-membered heterocyclyl having up to 2 heteroatoms from the group consisting of N, O and S represents a saturated heterocycle which is attached via a ring carbon atom or, if appropriate, via a ring nitrogen atom of the heterocycle. The following radicals may be mentioned by way of example and by way of preference: tetrahydrofuryl, pyrrolidinyl, piperidinyl, piperazinyl, morpholinyl and thiomorpholinyl.

In the context of the invention, halogen includes fluorine, chlorine, bromine and iodine. Preference is given to chlorine or fluorine.

Depending on the substitution pattern, the compounds according to the invention can exist in stereoisomeric forms which are either like image and mirror image (enantiomers) or not like image and mirror image (diastereomers). The invention relates both to the enantiomers or diastereomers and to their respective mixtures. The racemic forms, like the diastereomers, can be separated in a known manner into the stereoisomerically uniform components.

Furthermore, certain compounds can be present in tautomeric forms. This is known to the person skilled in the art, and such compounds are likewise included in the scope of the invention.

The compounds according to the invention can also be present as salts. In the context of the invention, preference is given to physiologically acceptable salts.

Physiologically acceptable salts can be salts of the compounds according to the invention with inorganic or organic acids. Preference is given to salts with inorganic acids such as, for example, hydrochloric acid, hydrobromic acid, phosphoric acid or sulphuric acid, or to salts with organic carboxylic or sulphonic acids such as, for example, acetic acid, propionic acid, maleic acid, fumaric acid, malic acid, citric acid, tartaric acid, lactic acid, benzoic acid, or methanesulphonic acid, ethanesulphonic acid, benzenesulphonic acid, toluenesulphonic acid or naphthalenedisulphonic acid.

Physiologically acceptable salts can also be salts of the compounds according to the invention with bases, such as, for example, metal or ammonium salts. Preferred examples are alkali metal salts (for example sodium salts or potassium salts), alkaline earth metal salts (for example magnesium salts or calcium salts), and also ammonium salts which are derived from ammonia or organic amines, such as, for example, ethylamine, di- or triethylamine, ethyldiisopropylamine, monoethanolamine, di- or triethanolamine, dicyclohexylamine, dimethylaminoethanol, dibenzylamine, N-methylmorpholine, dihydroabietylamine, 1-ephenamine, methylpiperidine, arginine, lysine, ethylenediamine or 2-phenylethylamine.

The compounds according to the invention can also be present in the form of their solvates, in particular in the form of their hydrates.

Preference is given to compounds of the general formula (I) in which
A represents the group C—R¹¹ or represents N,
   where
   R¹¹ represents hydrogen or methyl,
X represents O or S,
R¹ represents phenyl or represents 5- or 6-membered heteroaryl having up to two heteroatoms from the group consisting of N, O and S, which radicals may for their part each be mono- or disubstituted by identical or different substituents selected from the group consisting of fluorine, chlorine, cyano, (C₁-C₄)-alkyl, (C₁-C₄)-alkoxy, phenoxy, benzyloxy, trifluoromethyl, trifluoromethoxy, vinyl, phenyl, benzyl, methylthio, methylsulphonyl, acetyl, propionyl, (C₁-C₄)-alkoxycarbonyl, amino, acetylamino, mono- and di-(C₁-C₄)-alkylamino,
R² and R³ are identical or different and independently of one another represent hydrogen or (C₁-C₄)-alkyl or together with the carbon atom to which they are attached form a 5- or 6-membered spiro-linked cycloalkyl ring,
R⁴ represents hydrogen or methyl,
R⁵ represents hydrogen, methyl or ethyl,
R⁶ represents hydrogen or methyl,
R⁷ represents hydrogen, (C₁-C₄)-alkyl, (C₁-C₄)-alkoxy, fluorine or chlorine,
R⁸ and R⁹ are identical or different and independently of one another represent hydrogen or methyl, and
R¹⁰ represents hydrogen.

Particular preference is given to compounds of the general formula (I) in which
A represents CH or N,
X represents O,
R¹ represents phenyl or represents pyridyl which for their part may each be mono- or disubstituted by identical or different substituents selected from the group consisting of fluorine, chlorine, methyl, tert-butyl, methoxy, trifluoromethyl, trifluoromethoxy, methylthio, amino and dimethylamino,
R² represents hydrogen or methyl,
R³ represents methyl, isopropyl or tert-butyl, or
R² and R³ together with the carbon atom to which they are attached form a spiro-linked cyclohexane ring,
R⁴ represents hydrogen or methyl,
R⁵ represents hydrogen, methyl or ethyl,
R⁶ represents hydrogen or methyl,
R⁷ represents methyl,
R⁸ and R⁹ each represent hydrogen, and
R¹⁰ represents hydrogen.

The general or preferred radical definitions listed above apply both to the end products of the formula (I) and, correspondingly, to the starting materials and intermediates required in each case for the preparation.

The individual radical definitions given in the respective combinations or preferred combinations of radicals are, independently of the respectively given combinations of radicals, also replaced by any radical definitions of other combinations.

Of particular importance are compounds of the formula (I-A) 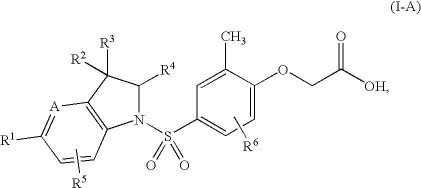
in which
R² represents hydrogen,
R³ represents methyl, isopropyl or tert-butyl, or
R² and R³ both represent methyl or together with the carbon atom to which they are attached form a spiro-linked cyclohexane ring, and
A, R¹, R⁴, R⁵ and R⁶ are each as defined above.

Moreover, we have found a process for preparing the compounds of the general formula (I) according to the invention, which process is characterized in that

compounds of the general formula (II)

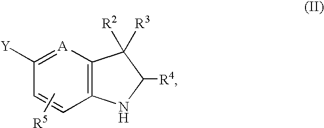

in which A, R², R³, R⁴ and R⁵ are each as defined above and

Y represents chlorine or bromine,

are initially converted using a compound of the general formula (III)

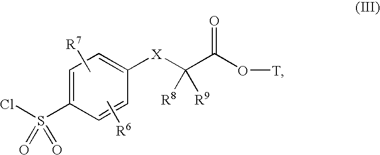

in which X, R⁶, R⁷, R⁸ and R⁹ are each as defined above and

T represents benzyl or (C₁-C₆)-alkyl,

in an inert solvent in the presence of a base into compounds of the general formula (IV)

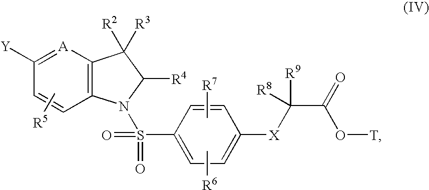

in which A, T, X, Y, R², R³, R⁴, R⁵, R⁶, R⁷, R⁸ and R⁹ are each as defined above,

these compounds are then reacted in a coupling reaction with a compound of the general formula (V) 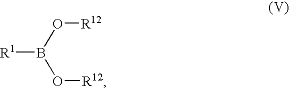
in which R¹ is as defined above and
R¹² represents hydrogen or methyl or both radicals together form a —CH₂CH₂— or —C(CH₃)₂—C(CH₃)₂— bridge,
in an inert solvent in the presence of a suitable palladium catalyst and a base to give compounds of the general formula (I-B)

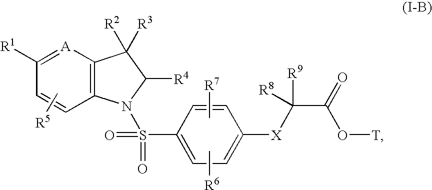

in which A, T, X, R¹, R², R³, R⁴, R⁵, R⁶, R⁷, R⁸ and R⁹ are each as defined above,

[cf., for example, W. Hahnfeld, M. Jung, Pharmazie 1994, 49, 18-20; idem, Liebigs Ann. Chem. 1994, 59-64],

the compounds (I-B) are then reacted with acids or bases or, if T represents benzyl, also hydrogenolytically, to give the corresponding carboxylic acids of the general formula (I-C)

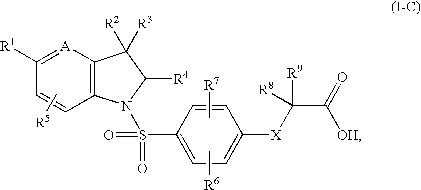

in which A, X, R¹, R², R³, R⁴, R⁵, R⁶, R⁷, R⁸ and R⁹ are each as defined above,

and the carboxylic acids (I-C) are, if appropriate, further modified by known esterification methods to give compounds of the general formula (I).

In the reaction sequence described above, the step of the coupling reaction [cf. (TV)+(V)→(I-B)] and the ester cleavage [cf. (I-B)→(I-C)] can optionally also be carried out in reverse order; in the coupling reaction, it is also possible to carry out a basic ester cleavage in situ.

Inert solvents for process step (II)+(III)→(IV) are, for example, halogenated hydrocarbons, such as dichloromethane, trichloromethane, carbon tetrachloride, trichloroethane, tetrachloroethane, 1,2-dichloroethane or trichloroethylene, ethers, such as diethyl ether, dioxane, tetrahydrofuran, glycol dimethyl ether or diethylene glycol dimethyl ether, hydrocarbons, such as benzene, xylene, toluene, hexane, cyclohexane or mineral oil fractions, or other solvents, such as nitromethane, ethyl acetate, acetone, dimethylformamide, dimethyl sulphoxide, acetonitrile, N-methylpyrrolidinone or pyridine. It is also possible to use mixtures of the solvents mentioned. Preference is given to dichloromethane or tetrahydrofuran.

Suitable bases for process step (II)+(E)→(IV) are the customary inorganic or organic bases. These preferably include alkali metal hydroxides such as, for example, lithium hydroxide, sodium hydroxide or potassium hydroxide, alkali metal or alkaline earth metal carbonates, such as sodium carbonate, potassium carbonate or calcium carbonate, alkali metal hydrides, such as sodium hydride, or organic amines, such as pyridine, triethylamine, ethyldiisopropylamine, N-methylmorpholine or N-methylpiperidine. Particular preference is given to amine bases such as triethylamine, pyridine or ethyldiisopropylamine, if appropriate in the presence of catalytic amounts (about 10 mol %) of 4-N,N-dimethylaminopyridine or 4-pyrrolidinopyridine.

Here, the base is employed in an amount of from 1 to 5, preferably 1 to 2.5, mol per mole of the compound of the general formula (III).

The reaction is generally carried out in a temperature range of from −20° C. to +100° C., preferably from 0° C. to +75° C. The reaction can be carried out at atmospheric, elevated or reduced pressure (for example from 0.5 to 5 bar). In general, the reaction is carried out at atmospheric pressure.

Inert solvents for process step (IV)+(V)→(I-B) are, for example, ethers, such as diethyl ether, dioxane, tetrahydrofuran, glycol dimethyl ether or diethylene glycol dimethyl ether, alcohols, such as methanol, ethanol, n-propanol, isopropanol, n-butanol or tert-butanol, hydrocarbons, such as benzene, xylene, toluene, hexane, cyclohexane or mineral oil fractions, or other solvents, such as dimethylformamide, acetonitrile or else water. It is also possible to use mixtures of the solvents mentioned. Preference is given to toluene, dimethylformamide or acetonitrile.

Suitable bases for process step (IV)+(V)→(I-B) are the customary inorganic or organic bases. These preferably include alkali metal hydroxides, such as, for example, lithium hydroxide, sodium hydroxide or potassium hydroxide, alkali metal or alkaline earth metal carbonates, such as sodium carbonate, potassium carbonate or calcium carbonate, alkali metal phosphates, such as sodium phosphate or potassium phosphate, or organic amines, such as pyridine, triethylamine, ethyldiisopropylamine, N-methylmorpholine or N-methylpiperidine. Particular preference is given to sodium carbonate or potassium carbonate or potassium phosphate.

Here, the base is employed in an amount of from 1 to 5, preferably from 2 to 3, mol per mole of the compound of the general formula (IV).

Suitable palladium catalysts for process step (IV)+(V)→(I-B) are, preferably, palladium(0) or palladium(II) compounds which are used in preformed form, such as, for example, [1,1′-bis(diphenylphosphino)ferrocenyl]palladium(II) chloride or bis(triphenylphosphine)palladium(II) chloride, or which may be generated in situ from a suitable palladium source, such as, for example, bis(dibenzylideneacetone)palladium(0) or tetrakis(triphenylphosphine)palladium(0), and a suitable phosphine ligand.

The reaction is generally carried out in a temperature range of from 0° C. to +150° C., preferably from +20° C. to +100° C. The reaction can be carried out at atmospheric, elevated or reduced pressure (for example from 0.5 to 5 bar). In general, the reaction is carried out at atmospheric pressure.

Inert solvents for process step (I-B)→(I-C) are, for example, halogenated hydrocarbons, such as dichloromethane, 1,2-dichloroethane or trichloroethylene, ethers, such as diethyl ether, dioxane, tetrahydrofuran, glycol dimethyl ether or diethylene glycol dimethyl ether, alcohols, such as methanol, ethanol, n-propanol, isopropanol, n-butanol or tert-butanol, hydrocarbons, such as benzene, xylene, toluene, hexane, cyclohexane or mineral oil fractions, or other solvents, such as nitromethane, acetone, dimethylformamide, dimethyl sulphoxide, acetonitrile or N-methylpyrrolidinone. It is also possible to use mixtures of the solvents mentioned. Preference is given to alcohols such as methanol or ethanol.

Suitable bases for process step (I-B)→(I-C) are the customary inorganic bases. These preferably include alkali metal hydroxides, such as, for example, lithium hydroxide, sodium hydroxide or potassium hydroxide, or alkali metal or alkaline earth metal carbonates, such as sodium carbonate, potassium carbonate or calcium carbonate. Particular preference is given to lithium hydroxide or sodium hydroxide.

Here, the base is employed in an amount of from 1 to 5, preferably from 1 to 3, mol per mole of the compound of the general formula (I-B).

Suitable acids for process step (I-B)→(I-C) are the customary inorganic acids, such as, for example, hydrochloric acid or sulphuric acid, or sulphonic acids, such as toluenesulphonic acid, methanesulphonic acid or trifluoromethanesulphonic acid, or carboxylic acids, such as trifluoroacetic acid.

In general, the reaction is carried out in a temperature range of from −20° C. to +100° C., preferably from 0° C. to +30° C. The reaction can be carried out at atmospheric, elevated or reduced pressure (for example from 0.5 to 5 bar). In general, the reaction is carried out at atmospheric pressure.

The compounds of the general formula (II) are known or can be prepared analogously to processes known from the literature by initially converting compounds of the general formula (VI)

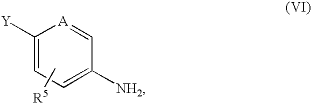

in which A, Y and R⁵ are each as defined above,

with sodium nitrite and tin(II) chloride in the presence of an acid into hydrazine derivatives of the general formula (VII)

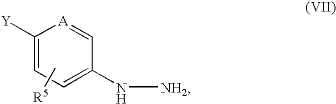

in which A, Y and R⁵ are each as defined above,

then reacting these in the presence of an acid or Lewis acid, if appropriate in an inert solvent, with a compound of the general formula (VIII)

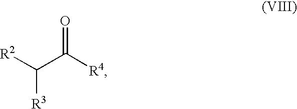

in which R², R³ and R⁴ are each as defined above,

if R² and R³ in (VIII) are both not hydrogen, to compounds of the general formula (IX), or, if R³ in (VIII) represents hydrogen, to compounds of the general formula (X)

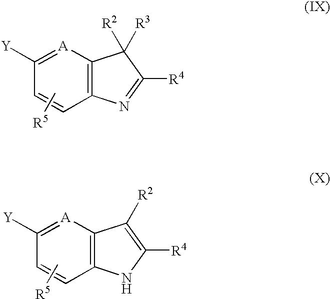

in which A, Y, R⁴ and R⁵ are each as defined above,

and then reducing the compounds (IX) or (X) with the aid of a borohydride, aluminium hydride or silicon hydride, such as, for example, sodium borohydride or sodium cyanoborohydride, or by hydrogenation in the presence of a suitable catalyst, such as, for example, Raney nickel [for process steps (VII)+(VIII)→(IX)→(II) cf., for example, P. E. Maligres, I. Houpis, K. Rossen, A. Molina, J. Sager, V. Upadhyay, K. M. Wells, R. A. Reamer, J. E. Lynch, D. Askin, R. P. Volante, P. J. Reider, Tetrahedron 1997, 53, 10983-10992].

Inert solvents for process step (VI)→(VII) are, for example, ethers, such as dioxane, glycol dimethyl ether or diethylene glycol dimethyl ether, alcohols, such as methanol, ethanol, n-propanol, iso-propanol, n-butanol or tert-butanol, or other solvents, such as dimethylformamide, dimethyl sulphoxide, N-methylpyrrolidinone or water. It is also possible to use mixtures of the solvents mentioned. The preferred solvent is water.

Suitable acids for process step (VI)→(VII) are the customary inorganic or organic acids. These preferably include hydrochloric acid, sulphuric acid or phosphoric acid, or carboxylic acids, such as formic acid, acetic acid or trifluoroacetic acid, or sulphonic acids, such as toluenesulphonic acid, methanesulphonic acid or trifluoromethanesulphonic acid. Particular preference is given to semiconcentrated to concentrated aqueous hydrochloric acid which simultaneously acts as solvent.

The reaction is generally carried out in a temperature range of from −30° C. to +80° C., preferably from −10° C. to +25° C. The reaction can be carried out at atmospheric, elevated or reduced pressure (for example from 0.5 to 5 bar). In general, the reaction is carried out at atmospheric pressure.

Inert solvents for process step (VII)+(VIII)→(IX) or (X) are, for example, halogenated hydrocarbons, such as dichloromethane, trichloromethane, carbon tetrachloride, trichloroethane, tetrachloroethane, 1,2-dichloroethane or trichloroethylene, ethers, such as dioxane, tetrahydrofuran, glycol dimethyl ether or diethylene glycol dimethyl ether, alcohols, such as methanol, ethanol, n-propanol, isopropanol, n-butanol or tert-butanol, or hydrocarbons, such as benzene, xylene, toluene, hexane, cyclohexane or mineral oil fractions, or other solvents, such as acetonitrile or water. It is also possible to use mixtures of the solvents mentioned. It is also possible to carry out the reaction without any solvent. If R³ represents hydrogen and A represents CH or N, the reaction is preferably carried out without any solvent to give the product (X); if R² and R³ are both not hydrogen and A represents CH, the reaction is preferably carried out in a mixture of toluene and acetonitrile to give the product (IX).

Suitable acids for process step (VII)+(VIII)→(IX) or (X) are the customary inorganic or organic acids. These preferably include hydrochloric acid, sulphuric acid or phosphoric acid, or carboxylic acids, such as formic acid, acetic acid or trifluoroacetic acid, or sulphonic acids, such as toluenesulphonic acid, methanesulphonic acid or trifluoromethanesulphonic acid. Alternatively, the customary Lewis acids, such as, for example, boron trifluoride, aluminium trichloride or zinc chloride are also suitable. Here, the acid is employed in an amount of from 1 to 10 mol per mole of the compound of the general formula (VII). If R³ represents hydrogen and A represents CH or N, the reaction is preferably carried out using 1 to 2 mol of zinc chloride to give the product (X), and if R² and R³ are both not hydrogen and A represents CH, the reaction is preferably carried out using 2 to 5 mol of trifluoroacetic acid to give the product (IX).

The reaction is generally carried out in a temperature range of from 0° C. to +250° C. If R³ represents hydrogen and A represents CH or N, the reaction is preferably carried out in a temperature range of from +130° C. to +200° C. to give the product (X); if R² and R³ are both not hydrogen and A represents CH, the reaction is preferably carried out in a temperature range of from 0° C. to +50° C. to give the product (IX). The reaction can be carried out at atmospheric, elevated or reduced pressure (for example from 0.5 to 5 bar). In general, the reaction is carried out at atmospheric pressure.

Reducing agents suitable for process step (IX) or (X)→(II) are borohydrides, aluminium hydrides or silicon hydrides, such as, for example, borane, diborane, sodium borohydride, sodium cyanoborohydride, lithium aluminium hydride or triethylsilane, if appropriate in the presence of an acid or Lewis acid, such as, for example, acetic acid, trifluoroacetic acid, aluminium trichloride or boron trifluoride, or hydrogenation with hydrogen in the presence of a suitable catalyst, such as, for example, palladium on activated carbon, platinum oxide or Raney nickel. In the case of compounds of the general formula (X) in which A represents N, preference is given to hydrogenation using Raney nickel as catalyst, and if A in (X) represents CH, preference is given to reduction with sodium cyanoborohydride. In the case of compounds of the general formula (IX), preference is given to using sodium borohydride.

Suitable solvents for process step (IX) or (X)→(II) are, for example, ethers, such as diethyl ether, dioxane, tetrahydrofuran, glycol dimethyl ether or diethylene glycol dimethyl ether, alcohols, such as methanol, ethanol, n-propanol, isopropanol, n-butanol or tert-butanol, or hydrocarbons, such as benzene, xylene, toluene, hexane, cyclohexane or mineral oil fractions, or other solvents, such as acetonitrile, acetic acid or water. It is also possible to use mixtures of the solvents mentioned. For the hydrogenation of the compounds of the general formula (X) in which A represents N, preference is given to using ethanol, and for the reduction in the case where A in (X) represents CH, preference is given to using acetic acid, a large excess of which is added as acid to the reducing agent and simultaneously serves as solvent. For the reduction of the compounds of the general formula (IX), preference is given to using a mixture of methanol and toluene/acetonitrile [from the reaction (VII)→(IX), with addition of 2 to 5 mol of trifluoroacetic acid] in a ratio of from 1:1 to 1:10.

The reaction is generally carried out in a temperature range of from −20° C. to +200° C. Here, the hydrogenation of the compounds (X) in which A represents N is preferably carried out in a temperature range of from +150° C. to +200° C., whereas the reduction of the compounds (IX) and (X) in which A represents CH is preferably carried out in a temperature range of from −10° C. to +50° C. The reaction can be carried out at atmospheric, elevated or reduced pressure (for example from 0.5 to 150 bar). Whereas the hydrogenation of the compounds (X) in which A represents N is preferably carried out in a pressure range of from 50 to 150 bar of hydrogen, the reduction of the compounds (IX) or (X) in which A represents CH is generally carried out at atmospheric pressure.

The compounds of the general formula (III) are known or can be prepared analogously to processes known from the literature, for example by initially converting a compound of the general formula (XI) 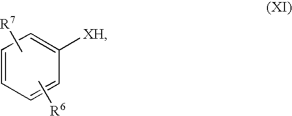
in which R⁶, R⁷ and X are each as defined above,

with a compound of the general formula (XII)

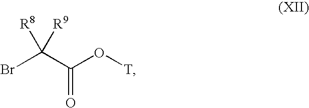

in which R⁸, R⁹ and T are each as defined above,

in an inert solvent in the presence of a base into a compound of the general formula (XIII)

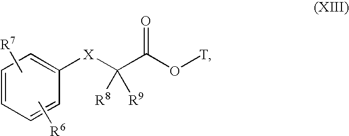

in which R⁶, R⁷, R⁸, R⁹, X and T are each as defined above,

and then reacting this compound with chlorosulphonic acid [cf., for example, P. D. Edwards, R. C. Mauger, K. M. Cottrell, F. X. Morris, K. K. Pine, M. A. Sylvester, C. W. Scott, S. T. Furlong, Bioorg. Med. Chem. Lett. 2000, 10, 2291-2294].

Inert solvents for process step (XI)+(XII)→(XIII) are, for example, ethers, such as diethyl ether, dioxane, tetrahydrofuran, glycol dimethyl ether or diethylene glycol dimethyl ether, hydrocarbons, such as benzene, xylene, toluene, hexane, cyclohexane or mineral oil fractions, or other solvents, such as acetone, dimethylformamide, dimethyl sulphoxide, acetonitrile or N-methylpyrrolidinone. It is also possible to use mixtures of the solvents mentioned. Preference is given to dimethylformamide or acetone.

Suitable bases for process step (XI)+(XII)→(XIII) are the customary inorganic or organic bases. These preferably include alkali metal hydroxides, such as, for example, lithium hydroxide, sodium hydroxide or potassium hydroxide, alkali metal or alkaline earth metal carbonates, such as sodium carbonate, potassium carbonate or calcium carbonate, alkali metal hydrides, such as sodium hydride, or organic amines, such as pyridine, triethylamine, ethyldiisopropylamine, N-methylmorpholine or N-methylpiperidine. Particular preference is given to potassium carbonate.

Here, the base is employed in an amount of from 1 to 5, preferably from 1 to 2, mol per mole of the compound of the general formula (XI).

The reaction is generally carried out in a tempereature range of from −20° C. to +150° C., preferably from 0° C. to +80° C. The reaction can be carried out at atmospheric, elevated or reduced pressure (for example from 0.5 to 5 bar). In general, the reaction is carried out at atmospheric pressure.

The compounds of the general formulae (V), (VI), (VIII), (XI) and (XII) are commercially available, known from the literature or can be prepared analogously to processes known from the literature.

The process according to the invention can be illustrated by reactions scheme 1 and 2 below:

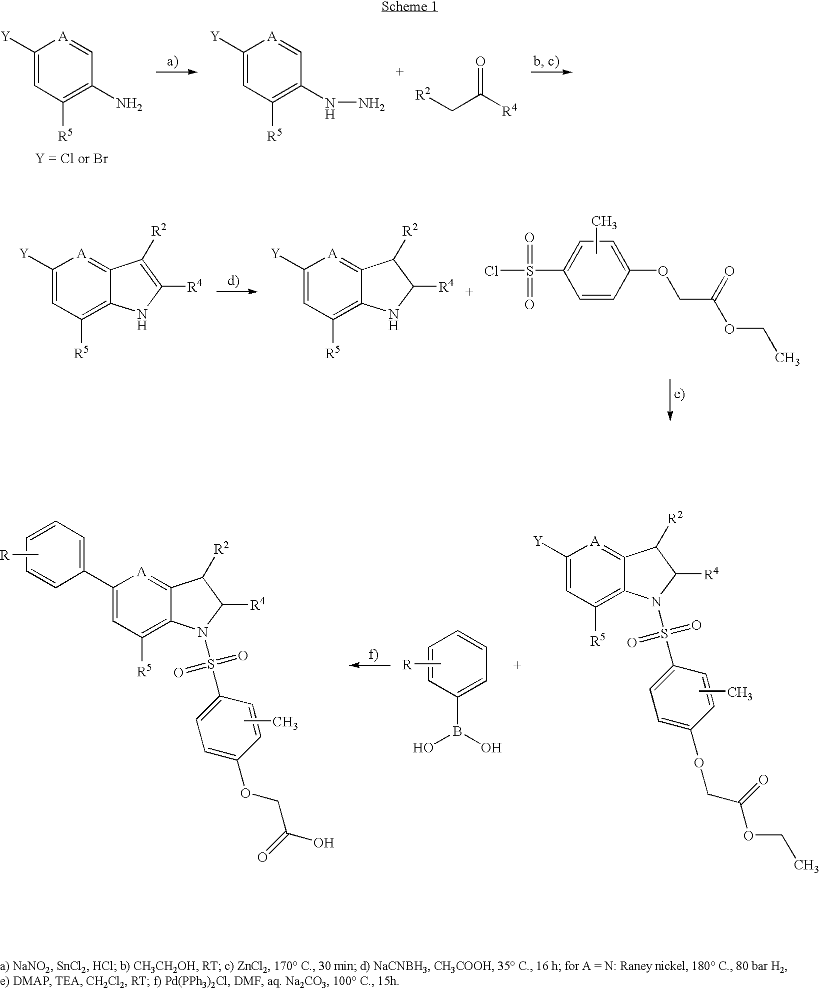

a) NaNO₂, SnCl₂, HCl; b) CH₃CH₂OH, RT; c) ZnCl₂, 170° C., 30 min; d) NaCNBH₃, CH₃COOH, 35° C., 16 h; for A=N: Raney nickel, 180° C., 80 bar H₂, e) DMAP, TEA, CH₂Cl₂, RT; f) Pd(PPh₃)₂Cl, DMF, aq. Na₂CO₃, 100° C., 15 h.

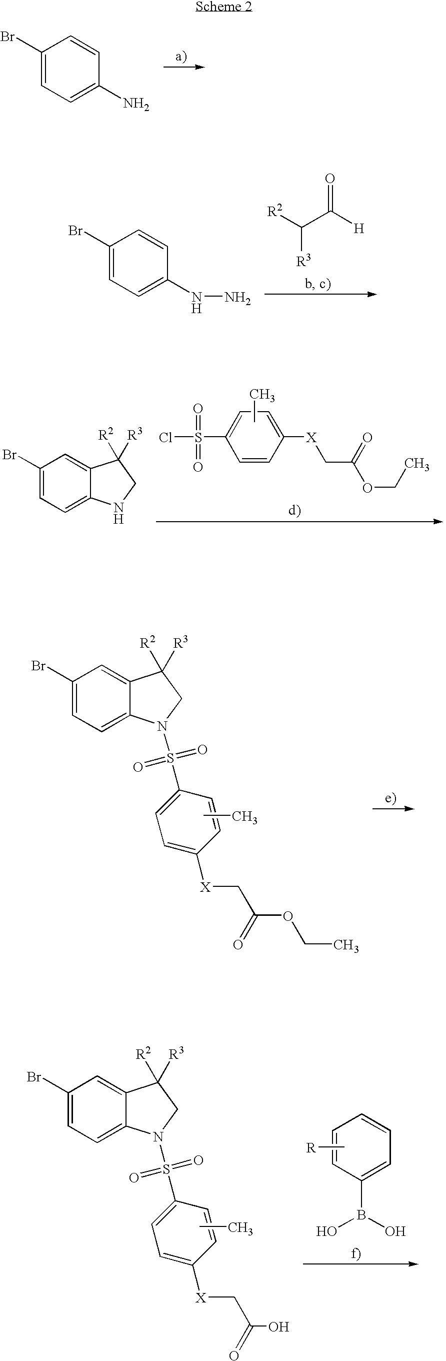

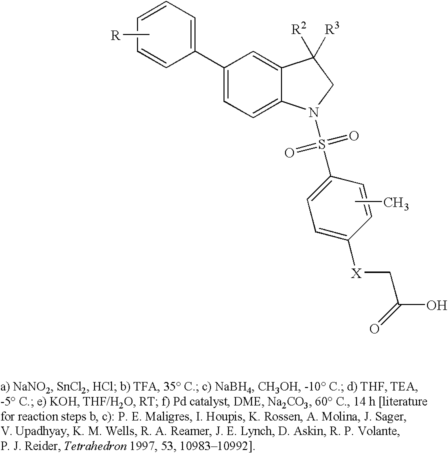

a) NaNO₂, SnCl₂, HCl; b) TFA, 35° C.; c) NaBH₄, CH₃OH, −10° C.; d) THF, TEA, −5° C.; e) KOH, THF/H₂O, RT; f) Pd catalyst, DME, Na₂CO₃, 60° C., 14 h [literature for reaction steps b, c): P. E. Maligres, I. Houpis, K. Rossen, A. Molina, J. Sager, V. Upadhyay, K. M. Wells, R. A. Reamer, J. E. Lynch, D. Askin, R. P. Volante, P. J. Reider, Tetrahedron 1997, 53, 10983-10992].

The compounds of the formula (I) according to the invention have a surprising and useful spectrum of pharmacological activity and can therefore be used as versatile medicaments, in particular for treating disorders in which the PPAR delta inhibitor is activiated. In particular, they are suitable for treating coronary heart disease, for the prophylaxis of myocardial infarction and for the treatment of restenosis after coronary angioplasty or stenting. The compounds of the formula (I) according to the invention are preferably suitable for treating stroke, CNS disorders, Alzheimer's, osteoporosis, arteriosclerosis and hypercholesterolaemia, for increasing pathologically low HDL levels and for lowering elevated triglyceride and LDL levels. In addition, they can be used for treating obesity, diabetes, for treating metabolic syndrome (glucose intolerance, hyperinsulinaemia, dyslipidaemia and high blood pressure owing to insulin resistance), hepatic fibrosis and cancer.

The novel active compounds can be administered alone or, if required, in combination with other active compounds, preferably from the group of the CETP inhibitors, antidiabetics, antioxidants, cytostatics, calcium antagonists, antihypertensives, thyroid hormones and/or thyroid mimetics, inhibitors of HMG-CoA reductase, inhibitors of HMG-CoA reductase expression, squalene synthesis inhibitors, ACAT inhibitors, perfusion promoters, platelet aggregation inhibitors, anticoagulants, angiotensin II receptor antagonists, cholesterol absorption inhibitors, MTP inhibitors, aldolase reductase inhibitors, fibrates, niacin, anorectics, lipase inhibitors and PPAR-α and/or PPAR-γ agonists. Further combinations with anti-inflammatory agents, for example COX-2 inhibitors, NEP inhibitors, ECE inhibitors, vasopeptidase inhibitors, aldose reduction inhibitors, antioxidants, cytostatics, perfusion promoters and anorectics are possible.

The compounds according to the invention are in each case preferably combined with an antidiabetic or a plurality of antidiabetics mentioned in the Rote Liste 2002/II, Chapter 12,
with one or more antithrombotics, by way of example and by way of preference from the group of the platelet aggregation inhibitors or the anticoagulants,
with one or more antihypertensives, by way of example and by way of preference from the group of the calcium antagonists, angiotensin AII antagonists, ACE inhibitors, beta blockers and the diuretics and/or
with one or more lipid metabolism-modifying active compounds from the group of the thyroid receptor agonists, cholesterol synthesis inhibitors such as, by way of example and by way of preference, HMG-CoA reductase or squalene synthesis inhibitors, ACAT inhibitors, MPT inhibitors, PPAR agonists, fibrates, cholesterol absorption inhibitors, lipase inhibitors, polymeric bile acid absorbers, lipoprotein(a) antagonists.

Antidiabetics are to be understood as meaning, by way of example or by way of preference, insulin and insulin derivatives, and also orally active hypoglycaemics.

Here, insulin and insulin derivatives include both insulins of animal, human or biotechnological origin, and also mixtures thereof.

The orally active hypoglycaemics include, by way of example and by way of preference, sulphonyl ureas, biguadines, meglitinide derivatives, oxoadiazolidinones, thiazolindinediones, glucosidase inhibitors, glucagon antagonists, GLP-1 agonists, insulin sensitizers, inhibitors of liver enzymes involved in the stimulation of gluconeogenesis and/or glycogenolysis, modulators of glucose uptake and potassium channel openers, for example those disclosed in WO 97/26265 and WO 99/03861 by Novo Nordisk A/S.

In a preferred embodiment of the invention, the compounds mentioned are administered in combination with insulin.

In a preferred embodiment of the invention, the compounds mentioned are administered in combination with a sulphonyl urea, such as, by way of example and by way of preference, tolbutamide, glibenclanide, glimepiride, glipizide or gliclazide.

In a preferred embodiment of the invention, the compounds mentioned are administered in combination with a biguanide, such as, by way of example and by way of preference, metformine.

In a preferred embodiment of the invention, the compounds mentioned are administered in combination with a meglitinde derivative, such as, by way of example and by way of preference, repaglinide or nateglinide.

In a preferred embodiment of the invention, the compounds mentioned are administered in combination with a PPAR gamma agonist, for example from the class of thiazolidinediones, such as, by way of example and by way of preference, pioglitazone or rosiglitazone.

In a preferred embodiment of the invention, the compounds mentioned are administered in combination with a mixed PPAR alpha/gamma agonist, such as, by way of example and by way of preference, GI-262570 (farglitazar), GW 2331, GW 409544, AVE 8042, AVE 8134, AVE 0847, MK-0767 (KRP-297), AZ-242.

Antithrombotics are to be understood as meaning, by way of preference, compounds from the group of the platelet aggregation inhibitors, such as, by way of example and by way of preference, aspirin, clopidogrel, ticlopidine, dipyridamole, or of the anticoagulants.

In a preferred embodiment of the invention, the compounds mentioned are administered in combination with a thrombin inhibitor, such as, by way of example and by way of preference, ximelagatran, melagatran, bivalirudin, clexane.

In a preferred embodiment of the invention, the compounds mentioned are administered in combination with a GPIIb-IIIa antagonist, such as, by way of example and by way of preference, tirofiban, abciximab.

In a preferred embodiment of the invention, the compounds mentioned are administered in combination with a factor Xa inhibitor, such as, by way of example and by way of preference, DX 9065a, DPC 906, JTV 803.

In a preferred embodiment of the invention, the compounds mentioned are administered in combination with heparin or low molecular weight heparin derivatives.

In a preferred embodiment of the invention, the compounds mentioned are administered in combination with a vitamin K antagonist, such as, by way of example and by way of preference, coumarin.

Antihypertensives are to be understood as meaning, by way of example and by way of preference, compounds from the group of the calcium antagonists, such as, by way of example and by way of preference, the compounds nipfedipine, verapamil, dilitazem, angiotensin, AII antagonists, ACE inhibitors, beta blockers, and also the diuretics.

In a preferred embodiment of the invention, the compounds mentioned are administered in combination with an antagonist of alpha 1 receptors.

In a preferred embodiment of the invention, the compounds mentioned are administered in combination with reserpine, minoxidil, diazoxide, dihydralazine, hydralazine, and also nitric oxide-releasing compounds, such as, by way of example and by way of preference, glycerol nitrate or nitroprusside sodium.

In a preferred embodiment of the invention, the compounds mentioned are administered in combination with an angiotensin AII antagonist, such as, by way of example and by way of preference, losartan, valsartan, telmisartan.

In a preferred embodiment of the invention, the compounds mentioned are administered in combination with an ACE inhibitor, such as, by way of example and by way of preference, enalapril, captopril.

In a preferred embodiment of the invention, the compounds mentioned are administered in combination with a beta blocker, such as, by way of example and by way of preference, propranolol, atenolol.

In a preferred embodiment of the invention, the compounds mentioned are administered in combination with a diuretic, such as, by way of example and by way of preference, furosemide.

Lipid metabolism-modifying agents are to be understood as meaning, by way of example and by way of preference, compounds from the group of the thyroid receptor agonists, cholesterols synthesis inhibitors, such as HMG-CoA reductase or squalene synthesis inhibitors, ACAT inhibitors, MTP inhibitors, PPAR agonists, fibrates, cholesterol absorption inhibitors, lipase inhibitors, polymeric bile acid absorbers, lipoprotein(a) antagonists.

In a preferred embodiment of the invention, the compounds mentioned are administered in combination with a thyroid receptor agonist, such as, by way of example and by way of preference, D-thyroxine, 3,5,3′-triiodothyronine (T3), CGS 23425, axitirome (CGS 26214).

In a preferred embodiment of the invention, the compounds mentioned are administered in combination with a squalene synthesis inhibitor, such as, by way of example and by way of preference, BMS-188494, TAK 457.

In a preferred embodiment of the invention, the compounds mentioned are administered in combination with an ACAT inhibitor, such as, by way of example and by way of preference, avasimibe.

In a preferred embodiment of the invention, the compounds mentioned are administered in combination with a cholesterol absorption inhibitor, such as, by way of example and by way of preference, ezetimibe, tiqueside, pamaqueside.

In a preferred embodiment of the invention, the compounds mentioned are administered in combination with an MTP inhibitor, such as, by way of example and by way of preference, implitapide, BMS-201038, R-103757.

In a preferred embodiment of the invention, the compounds mentioned are administered in combination with a PPAR alpha agonist, such as, for example, the fibrates fenobfibrate, clofibrate, bezafibrate, ciprofibrate, gemfibrozil or such as, by way of example and by way of preference, GW 9578, GW 7647, LY-518674 or NS-220.

In a preferred embodiment of the invention, the compounds mentioned are administered in combination a CEPT inhibitor, such as, by way of example and by way of preference, torcetrapib (CP-5239 414), JJT-705.

In a preferred embodiment of the invention, the compounds mentioned are administered in combination with a mixed PPAR alpha/gamma agonist, such as, by way of example and by way of preference, GI-262570 (farglitazar), GW 2331, GW 409544, AVE 8042, AVE 8134, AVE 0847, MK-0767 (KRP-297), AZ-242.

In a preferred embodiment of the invention, the compounds mentioned are administered in combination with a lipase inhibitor, such as, by way of example and by way of preference, orlistat.

In a preferred embodiment of the invention, the compounds mentioned are administered in combination with a polymeric bile acid adsorber, such as, by way of example and by way of preference, cholestyramine, colestipol, colesolvam, CholestaGel, colestimide.

In a preferred embodiment of the invention, the compounds mentioned are administered in combination with a lipoprotein(a) antagonist, such as, by way of example and by way of preference, gemcabene calcium (CI-1027) or nicotinic acid.

In a preferred embodiment of the invention, the compounds mentioned are administered in combination with an antagonist of the niacin receptor.

In a preferred embodiment of the invention, the compounds mentioned are administered in combination with an LDL receptor inducer.

The invention also provides combinations of the compounds of the formulae (I) to (III) with HMG-CoA reductase inhibitors from the class of the statins, such as, by way of example and by way of preference, lovastatin, simvastatin, pravastatin, fluvastatin, atorvastatin, rosuvastatin and cerivastatin, pitavastatin.

The activity of the compounds according to the invention can be examined, for example, in vitro by the transactivation assay described in the experimental section.

The activity of the compounds according to the invention in vivo can be examined, for example, by the tests described in the experimental section.

Suitable administration forms for administering the compounds of the general formula (I) are all customary administration forms, i.e. oral, parenteral, inhalative, nasal, sublingual, rectal, external, for example transdermal, or local, such as, for example, in the case of implants or stents. In the case of parenteral administration, particular mention has to be made of intravenous, intramuscular and subcutaneous administration, for example as a subcutaneous depot. Preference is given to oral or parenteral administration. Very particular preference is given to oral administration.

Here, the active compounds can be administered on their own or in the form of preparations. Preparations suitable for oral administration are, inter alia, tablets, capsules, pellets, sugar-coated tablets, pills, granules, solid and liquid aerosols, syrups, emulsions, suspensions and solutions. Here, the active compound has to be present in such an amount that a therapeutic effect is obtained. In general, the active compound can be present in a concentration of from 0.1 to 100% by weight, in particular from 0.5 to 90% by weight, preferably from 5 to 80% by weight. In particular, the concentration of active compound should be 0.5 to 90% by weight, i.e. the active compound should be present in amounts sufficient to reach the dosage range stated.

To this end, the active compounds can be converted in a manner known per se into the customary preparations. This is carried out using inert non-toxic pharmaceutically acceptable carriers, auxiliaries, solvents, vehicles, emulsifiers and/or dispersants.

Auxiliaries which may be mentioned are, for example: water, non-toxic organic solvents, such as, for example, paraffins, vegetable oils (for example sesame oil), alcohols (for example ethanol, glycerol), glycols (for example polyethylene glycol), solid carriers, such as natural or synthetic ground minerals (for example talc or silicates), sugar (for example lactose), emulsifiers, dispersants (for example polyvinylpyrrolidone) and glidants (for example magnesium sulphate).

In the case of oral administration, tablets may, of course, also contain additives such as sodium citrate, together with additives such as starch, gelatine and the like. Aqueous preparations for oral administration may furthermore comprise flavour improvers or colorants.

In the case of oral administration, preference is given to administering dosages of from 0.001 to 5 mg/kg, preferably from 0.005 to 3 mg/kg, of body weight per 24 hours.

The working examples below illustrate the invention. The invention is not limited to the examples.

LC/MS Methods:

Method A: column: Waters Symmetry C18 50×2.1 mm, 3.5 μm; 0.5 ml/min; A: acetonitrile+0.1% formic acid, B: water+0.1% formic acid; 0 min 10% A, 4 min 90% A; 40° C.

Method B: instrument: Finnigan MAT 900S, TSP: P4000, AS3000, UV3000HR; column: Symmetry C 18, 150 mm×2.1 mm, 5.0 μm; mobile phase C: water, mobile phase B: water+0.3 g/l 35% strength hydrochloric acid, mobile phase A: acetonitrile; gradient: 0.0 min 2% A→2.5 min 95% A→5 min 95% A; oven: 70° C.; flow rate: 1.2 ml/min; UV detection: 210 nm.

Method C: instrument: Micromass Quattro LCZ, HP1100; column: Symmetry C18, 50 mm×2.1 mm, 3.5 μm; mobile phase A: acetonitrile+0.1% formic acid, mobile phase B: water+0.1% formic acid; gradient: 0.0 min 10% A→4.0 min 90% A→6.0 min 90% A; oven: 40° C.; flow rate: 0.5 ml/min; UV detection: 208-400 nm.

Method D: instrument: Micromass Platform LCZ, HP1100; column: Symmetry C18, 50 mm×2.1 mm, 3.5 μm; mobile phase A: acetonitrile+0.1% formic acid, mobile phase B: water+0.1% formic acid; gradient: 0.0 min 10% A→4.0 min 90% A→6.0 min 90% A; oven: 40° C.; flow rate: 0.5 ml/min; UV detection: 208-400 nm.

Method E: instrument: Micromass Platform LCZ, HP1100; column: Symmetry C18, 50 mm×2.1 mm, 3.5 μm; mobile phase A: acetonitrile+0.5% formic acid, mobile phase B: water+0.5% formic acid; gradient: 0.0 min 90% A→4.0 min 10% A→6.0 min 10% A; oven: 50° C.; flow rate: 0.5 ml/min; UV detection: 208-400 nm.

Method F: instrument: Micromass TOF-MUX-Interface/Waters600; column: YMC-ODS AQ, 50 mm×2.1 mm, 3.5 μm; temperature: 20° C.; flow rate: 0.8 ml/min; mobile phase A: acetonitrile+0.05% formic acid, mobile phase B: water+0.05% formic acid; gradient: 0.0 min 0% A→0.2 min 0% A→2.9 min 70% A→3.1 min 90% A.

GC/MS:

| | |
|---|---|
| Carrier gas: | helium |
| Flow rate: | 1.5 ml/min |
| Initial temperature: | 60° C. |
| Temperature gradient: | 14° C./min to 300° C., then 1 min const. 300° C. |
| Column: | HP-5 30 m × 320 μm × 0.25 μm (film thickness) |
| Initial time: | 2 min |
| Front injector temp.: | 250° C. |

### ABBREVIATIONS USED

abs. absolute
aq. aqueous
DMAP 4-N,N-dimethylaminopyridine
DME 1,2-dimethoxyethane
DMF N,N-dimethylformamide
DMSO dimethyl sulphoxide
ESI electrospray ionization (MS)
GC gas chromatography
LC-MS liquid chromatography-coupled mass spectroscopy
MS mass spectroscopy
MW molecular weight
NMR nuclear magnetic resonance spectroscopy
R_{f} retention index (TLC)
RT room temperature
Rₜ retention time (HPLC)
TEA triethylamine
TFA trifluoroacetic acid
THF tetrahydrofuran

### WORKING EXAMPLES

### Example 1

### [4-({3-Isopropyl-7-methyl-5-[4-(trifluoromethyl)phenyl]-2,3-dihydro-1H-indol-1-yl}-sulphonyl)-2-methylphenoxy]acetic acid

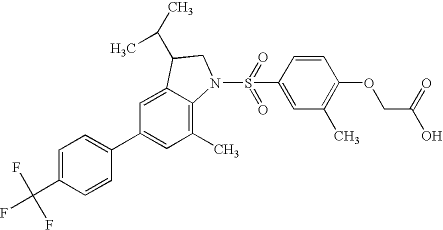 Step a):

### 1-(4-Bromo-2-methylphenyl)hydrazine

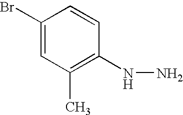

In 190 ml of concentrated hydrochloric acid, 50 g (267.7 mmol) of 4-bromo-2-methylaniline are heated at 80° C. for 30 min. After cooling to 5° C., 18.5 g (267.7 mmol) of sodium nitrite in 95 ml of water are added dropwise over a period of 30 min. After 30 minutes of stirring at 5° C., the reaction mixture is added dropwise over a period of 45 min to a solution of 384 g (2 mol) of tin chloride in 190 ml of concentrated hydrochloric acid. After a further 45 min at RT, the suspension is made alkaline using 50% strength aqueous sodium hydroxide solution. The precipitate is filtered off and extracted repeatedly with dichloromethane and ethyl acetate. The combined organic phases are dried over magnesium sulphate and concentrated. This gives 43.6 g (81% of theory) of the product as beige crystals.

LC-MS (method B): Rₜ=2.06 min

MS (ESIpos): m/z=201 (M+H)⁺

Step b):

### 5-Bromo-3-isopropyl-7-methyl-1H-indole

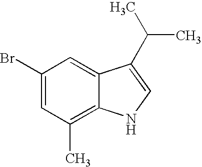

7 g (34.8 mmol) of 1-(4-bromo-2-methylphenyl)hydrazine are suspended in 14 ml of ethanol, and 3.9 g (45 mmol) of isovaleraldehyde are added. The mixture is stirred at RT for 30 minutes and the solvent is then removed under reduced pressure and the intermediate is, without further purification, melted at 170° C. with 5.2 g (38 mmol) of anhydrous zinc chloride. After 30-45 min, the melt is cooled to RT, taken up in dichloromethane and extracted with dilute hydrochloric acid and water. The organic phase is dried over magnesium sulphate and the solvent is removed under reduced pressure. The crude product is dissolved in ethyl acetate and purified on silica gel (mobile phase: cyclohexane/ethyl acetate 9:1). This gives 4.2 g (48% of theory).

LC-MS (method B): Rₜ=3.15 min

MS (ESIpos): m/z=253 (M+H)⁺

¹H-NMR (300 MHz, acetone-d₆): δ=1.51 (d, 6 H), 2.67 (s, 3H), 3.37 (m, 1H), 7.23 (s, 1H), 7.34 (s, 1H), 7.78 (s, 1H), 10.28 (s, 1H).

Step c):

### 5-Bromo-3-isopropyl-7-methylindoline

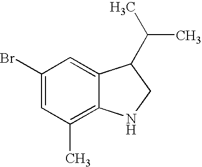

4.1 g (16.3 mmol) of 5-bromo-3-isopropyl-7-methyl-1H-indole are dissolved in 30 ml of glacial acetic acid and, at RT, 5.1 g (81 mmol) of sodium cyanoborohydride are added a little at a time. The reaction mixture is warmed at 35° C. for 16 hours and then hydrolysed with water and extracted twice with ethyl acetate. The extract is dried over sodium sulphate and the solvent is then removed under reduced pressure. The crude product is dissolved in ethyl acetate and purified on silica gel (mobile phase: cyclohexane/ethyl acetate 9:1). This gives 1.6 g (39% of theory).

LC-MS (method C): Rₜ=4.27 min

MS (ESIpos): m/z=255 (M+H)⁺

¹H-NMR (300 MHz, acetone-d₆): δ=0.85 (d, 3 H), 0.97 (d, 3H), 2.04 (m, 1H), 2.81 (s, 3H), 3.25 (m, 1H), 3.42 (dd, 1H), 3.58 (m, 1H), 6.96 (s, 1H), 7.02 (s, 1H).

Step d):

### Ethyl 2-methylphenoxyacetate

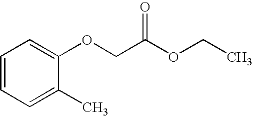

10.81 g (0.10 mol) of 2-methylphenol and 13.82 g (0.10 mol) of potassium carbonate are suspended in 100 ml of N,N-dimethylformamide and stirred at 50° C. for 1 hour. 18.37 g (0.11 mol) of ethyl bromoacetate are then added dropwise and the mixture is stirred at 50° C. overnight. After cooling to room temperature, the mixture is concentrated under reduced pressure, taken up in ethyl acetate and washed three times with water. The organic phase is dried over sodium sulphate and the solvent is removed under reduced pressure. Kugelrohr distillation of the residue gives 18.5 g (95% of theory) of the desired product.

GC-MS: Rₜ=12.50 min.

MS (ESIpos): m/z=194 (M)⁺

¹H-NMR (300 MHz, CDCl₃): δ=1.29 (t, 3H), 2.29 (s, 3H), 4.26 (q, 2H), 4.62 (s, 2H), 6.70 (d, 1H), 6.89 (dt, 1H), 7.22 (t, 1H), 7.25 (d, 1H).

Step e):

### Ethyl [4-(chlorosulphonyl)-2-methylphenoxy]acetate

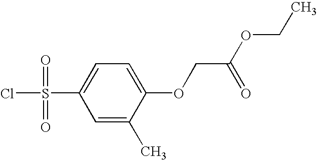

110 g (0.5 mol) of ethyl (2-methylphenoxy)acetate are initially charged in 250 ml of chloroform and cooled to 0° C. 330 g (2.8 mol) of chlorosulphonic acid are slowly added dropwise to the solution. The reaction mixture is stirred at RT for four hours and then poured onto ice and extracted three times with dichloromethane. The organic phase is washed twice with water, once with saturated sodium bicarbonate solution and once with saturated sodium chloride solution. The mixture is dried over sodium sulphate and the solvent is then removed under reduced pressure. This gives 153 g (93% of theory).

LC-MS (method C): Rₜ=3.95 min

MS (ESIpos): m/z=293 (M+H)⁺

¹H-NMR (300 MHz, CDCl₃): δ=1.31 (t, 3H), 2.36 (s, 3H), 4.28 (q, 2H), 4.75 (s, 2H), 6.81 (m, 2H), 7.85 (m, 2H).

Step f):

### Ethyl {4-[(5-bromo-3-isopropyl-7-methyl-2,3-dihydro-1H-indol-1-yl)sulphonyl]-2-methylphenoxy}acetate

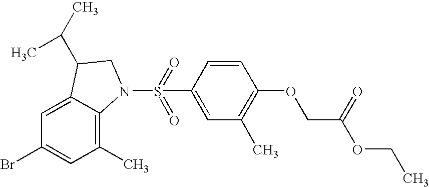

2.5 g (9.8 mmol) of 5-bromo-3-isopropyl-7-methylindoline are dissolved in 20 ml of tetrahydrofuran, and 3 ml (21 mmol) of triethylamine, 20 mg (0.16 mmol) of DMAP and 2.8 g (9.8 mmol) of ethyl [4-(chlorosulphonyl)-2-methylphenoxy]acetate are added. The reaction mixture is stirred at RT overnight. The mixture is filtered and the solvent is then removed under reduced pressure and the crude product is purified on silica gel (mobile phase: cyclohexane/ethyl acetate 9:1). This gives 4.8 g (96% of theory).

LC-MS (method B): Rₜ=3.29 min

MS (ESIpos): m/z=510 (M+H)⁺

¹H-NMR (300 MHz, CDCl₃): δ=0.62 (d, 3H), 0.82 (d, 3H), 1.29 (t, 3H), 1.84 (m, 1H), 2.22 (s, 3H), 2.27 (m, 1H), 2.51 (s, 3H), 3.56 (dd, 1H), 3.95 (dd, 1H), 4.27 (q, 2H), 4.68 (s, 2H), 6.62 (m, 1H), 6.69 (s, 1H), 7.25 (s, 1H), 7.30 (m, 2H).

Step g):

### [4-({3-Isopropyl-7-methyl-5-[4-(trifluoromethyl)phenyl]-2,3-dihydro-1H-indol-1yl]-sulphonyl)-2-methylphenoxy]acetic acid

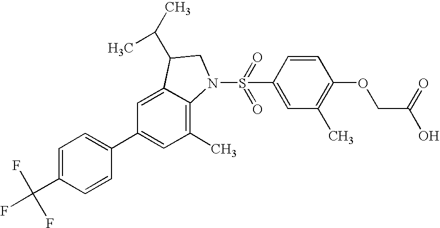

0.1 g (0.19 mmol) of ethyl {4-[(5-bromo-3-isopropyl-7-methyl-2,3-dihydro-1H-indol-1-yl)sulphonyl]-2-methylphenoxy}acetate is dissolved in 6 ml of absolute dimethylformamide, and 7 mg (0.01 mmol) of bis(triphenylphosphine)palladium(II) chloride and 48.3 mg (0.25 mmol) of 4-trifluoromethylphenylboronic acid are added under argon. The mixture is stirred at 70° C. for 30 minutes, and 1 ml of a 2 M solution of sodium carbonate is then added. The reaction mixture is heated at 100° C. for 16 h. After cooling to RT, the mixture is filtered through silica gel. The solvent is removed under reduced pressure and the crude product is purified by preparative HPLC (YMC gel ODS-AQ S 5/15 μm; mobile phase A: water, mobile phase B: acetonitrile, gradient 0 min 30% B, 5 min 30% B, 50 min 95% B). This gives 65 mg (60% of theory).

LC-MS (method B): Rₜ=3.25 min

MS (ESIpos): m/z=548 (M+H)⁺

¹H-NMR (300 MHz, CDCl₃): δ=0.80 (d, 3H), 1.86 (m, 1H), 2.22 (s, 3H), 2.31 (m, 1H), 2.50 (s, 3H), 3.58 (dd, 1H), 3.95 (dd, 1H), 4.69 (s, 2H), 6.59 (m, 1H), 6.69 (s, 1H), 7.28 (s, 1H), 7.33 (m, 2H).

### Example 2

### [2-Methyl-4-({2,3,7-trimethyl-5-[4-(trifluoromethyl)phenyl]-2,3-dihydro-1H-indol-1-yl}sulphonyl)phenoxy]acetic acid

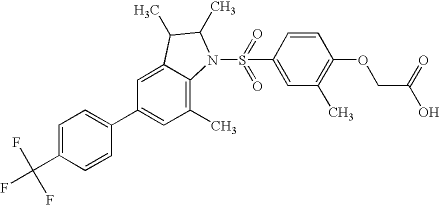 Step a):

### 5-Bromo-2,3,7-trimethyl-1H-indole

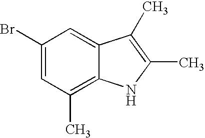

8 g (39.8 mmol) of 1-(4-bromo-2-methylphenyl)hydrazine (Example 1/step a) are suspended in 14 ml of ethanol, and 3.7 g (52 mmol) of ethyl methyl ketone are added. After 30 minutes of stirring at RT, the solvent is removed under reduced pressure and the intermediate is, without further purification, melted at 170° C. with 5.9 g (43 mmol) of anhydrous zinc chloride. After 30-45 min, the melt is cooled to RT, taken up in dichloromethane and extracted with dilute hydrochloric acid and water. The organic phase is dried over magnesium sulphate and the solvent is removed under reduced pressure. The crude product is dissolved in ethyl acetate and purified on silica gel (mobile phase: cyclohexane/ethyl acetate 9:1). This gives 3.8 g (40% of theory).

LC-MS (method D): Rₜ=4.92 min

MS (ESIpos): m/z=238 (M+H)⁺

¹H-NMR (300 MHz, acetone-d₆): δ=2.24 (s, 3H), 2.43 (s, 3H), 2.52 (s, 3H), 7.03 (s, 1H), 7.45 (s, 1H), 9.96 (s, 1H).

Step b):

### 5-Bromo-2,3,7-trimethylindoline

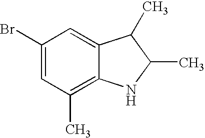

3.8 g (15.8 mmol) of 5-bromo-3,7-dimethyl-1H-indole are dissolved in 30 ml of glacial acetic acid and, at RT, 5 g (80 mmol) of sodium cyanoborohydride are added a little at a time. The reaction mixture is warmed at 35° C. for 16 hours and then hydrolysed with water and extracted twice with ethyl acetate. After drying over sodium sulphate, the solvent is removed under reduced pressure. The crude product is dissolved in ethyl acetate and purified on silica gel (mobile phase: cyclohexane/ethyl acetate 9:1). This gives 1.4 g (37% of theory).

LC-MS (method B): Rₜ=2.66 min

MS (ESIpos): m/z=240 (M+H)⁺

¹H-NMR (300 MHz, CDCl₃): δ=1.26 (d, 3 H), 1.32 (d, 3H), 2.08 (s, 3H), 2.85 (m, 1H), 3.48 (m, 1H), 6.98 (s, 2H).

Step c):

### Ethyl {4-[(5-bromo-2,3,7-trimethyl-2,3-dihydro-1H-indol-1-yl)sulphonyl]-2-methyl-phenoxy}acetate

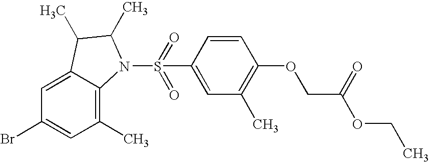

1.3 g (5.7 mmol) of 5-bromo-2,3,7-trimethylindoline are dissolved in 4 ml of tetrahydrofuran, and 1.7 ml (12.5 mmol) of triethylamine, 20 mg (0.16 mmol) of DMAP and 1.6 g (5.7 mmol) of ethyl [4-(chlorosulphonyl)-2-methylphenoxy]acetate (Example 1/step e) are added. The reaction mixture is stirred at RT overnight. Following filtration, the solvent is removed under reduced pressure and the crude product is purified on silica gel (mobile phase: cyclohexane/ethyl acetate 9:1). This gives 0.6 g (23% of theory).

LC-MS (method B): Rₜ=3.15 min

MS (ESIpos): m/z=496 (M+H)⁺

¹H-NMR (300 MHz, CDCl₃): δ=0.56 (d, 3H), 1.23 (d, 3H), 1.27 (t, 3H), 2.25 (s, 3H), 2.49 (m, 4H), 3.98 (m, 1H), 4.23 (q, 2H), 4.63 (s, 2H), 6.64 (d, 1H), 7.00 (m, 1H), 7.23 (m, 1H), 7.39 (m, 2H).

Step d):

### [2-Methyl-4-({2,3,7-trimethyl-5-[4-(trifluoromethyl)phenyl]-2,3-dihydro-1H-indol-1-yl}sulphonyl)phenoxy]acetic acid

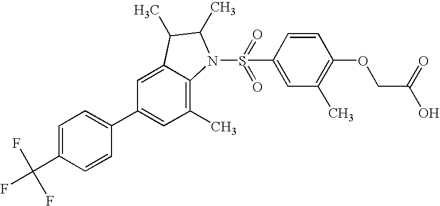

0.08 g (0.16 mmol) of ethyl {4-[(5-bromo-2,3,7-trimethyl-2,3-dihydro-1H-indol-1-yl)sulphonyl]-2-methylphenoxy}acetate is dissolved in 6 ml of absolute dimethylformamide, and 7 mg (0.01 mmol) of bis(triphenylphosphine)palladium(II) chloride and 40 mg (0.21 mmol) of 4-trifluoromethylphenylboronic acid are added under argon. The mixture is stirred at 70° C. for 30 minutes, and 1 ml of a 2 M solution of sodium carbonate is then added. The reaction mixture is heated at 100° C. for 16 h. After cooling to RT, the mixture is filtered through silica gel. The solvent is removed under reduced pressure and the crude product is purified by preparative HPLC (YMC gel ODS-AQ S 5/15 μm; mobile phase A: water, mobile phase B: acetonitrile, gradient 0 min 30% B, 5 min 30% B, 50 min 95% B). This gives 64 mg (74% of theory).

LC-MS (method C): Rₜ=5.26 min

MS (ESIpos): m/z=534 (M+H)⁺

¹H-NMR (300 MHz, CDCl₃): δ=0.61 (d, 3H), 0.8 (d, 3H), 2.61 (s, 3H), 3.57 (m, 1H), 3.78 (s, 2H), 3.91 (m, 1H), 6.51 (d, 1H), 6.90 (d, 2H), 6.98 (s, 1H), 7.18 (d, 2H), 7.40 (m, 3H).

### Example 3

### [4-({3,7-Dimethyl-5-[4-(trifluoromethyl)phenyl]-2,3-dihydro-1H-indol-1-yl}-sulphonyl)-2-methylphenoxy]acetic acid

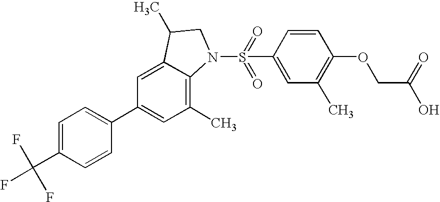 Step a):

### 5-Bromo-3,7-dimethyl-1H-indole

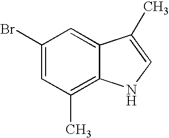

5 g (24.8 mmol) of 1-(4-bromo-2-methylphenyl)hydrazine (Example 1/step a) are suspended in 14 ml of ethanol, and 1.8 g (32 mmol) of propionaldehyde are added. The mixture is stirred at RT for 30 minutes and the solvent is then removed under reduced pressure and the intermediate is, without further purification, melted at 170° C. with 3.7 g (27 mmol) of anhydrous zinc chloride. After 30-45 min, the melt is cooled to RT, taken up in dichloromethane and extracted with dilute hydrochloric acid and water. The organic phase is dried over magnesium sulphate and the solvent is removed under reduced pressure. The crude product is dissolved in ethyl acetate and purified on silica gel (mobile phase: cyclohexane/ethyl acetate 9:1). This gives 1.5 g (27% of theory).

LC-MS (method C): Rₜ=4.65 min

MS (ESIpos): m/z=224 (M+H)⁺

¹H-NMR (300 MHz, acetone-d₆): δ=2.26 (s, 3H), 2.48 (s, 3H), 7.06 (s, 1H), 7.12 (s, 1H), 7.51 (s, 1H).

Step b):

### 5-Bromo-3,7-dimethylindoline

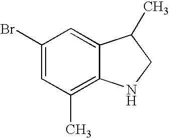

1.4 g (6.4 mmol) of 5-bromo-3,7-dimethyl-1H-indole are dissolved in 30 ml of glacial acetic acid, and 2 g (33 mmol) of sodium cyanoborohydride are added a little at a time at RT. The reaction mixture is warmed at 35° C. for 16 hours and then hydrolysed with water and extracted twice with ethyl acetate. After drying over sodium sulphate, the solvent is removed under reduced pressure. The crude product is dissolved in ethyl acetate and purified on silica gel (mobile phase: cyclohexane/ethyl acetate 9:1). This gives 0.79 g (53% of theory).

LC-MS (method B): Rₜ=2.38 min

MS (ESIpos): m/z=227 (M+H)⁺

¹H-NMR (300 MHz, CDCl₃): δ=1.29 (d, 3H), 2.09 (s, 3H), 3.13 (t, 1H), 3.36 (m, 1H), 3.72 (t, 1H), 6.99 (s, 1H), 7.03 (s, 1H).

Step c):

### Ethyl {4-[(5-bromo-3,7-dimethyl-2,3-dihydro-1H-indol-1-yl)sulphonyl]-2-methyl-phenoxy}acetate

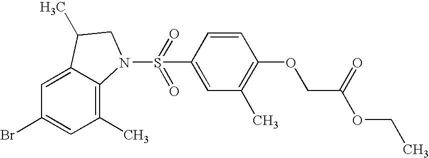

0.7 g (3.4 mmol) of 5-bromo-3,7-dimethylindoline is dissolved in 4 ml of tetrahydrofuran, and 1 ml (7.4 mmol) of triethylamine, 20 mg of DMAP and 1 g (3.4 mmol) of ethyl [4-(chlorosulphonyl)-2-methylphenoxy]acetate (Example 1/step e) are added. The reaction mixture is stirred at RT overnight. Following filtration, the solvent is removed under reduced pressure and the crude product is purified on silica gel (mobile phase: cyclohexane/ethyl acetate 9:1). This gives 1.5 g (90% of theory).

LC-MS (method D): Rₜ=5.25 min

MS (ESIpos): m/z=482 (M+H)⁺

¹H-NMR (300 MHz, CDCl₃): δ=0.98 (d, 3H), 1.28 (t, 3H), 2.22 (s, 3H), 2.39 (m, 1H), 2.52 (s, 3H), 3.31 (dd, 1H), 4.14 (dd, 1H), 4.27 (q, 2H), 4.66 (s, 2H), 6.61 (d, 1H), 6.93 (s, 1H), 7.26 (m, 3H).

Step d):

### [4-({3,7-Dimethyl-5-[4-(trifluoromethyl)phenyl]-2,3-dihydro-1H-indol-1-yl}-sulphonyl)-2-methylphenoxy]acetic acid

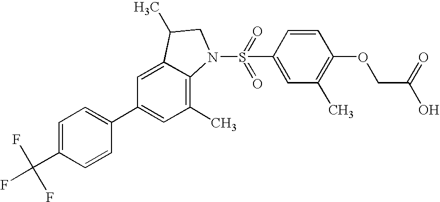

0.1 g (0.2 mmol) of ethyl {4-[(5-bromo-3,7-dimethyl-2,3-dihydro-1H-indol-1-yl)-sulphonyl]-2-methylphenoxy}acetate is dissolved in 6 ml of absolute dimethylformamide, and 7 mg (0.01 mmol) of bis(triphenylphosphine)palladium(II) chloride and 51 mg (0.26 mmol) of 4-trifluoromethylphenylboronic acid are added under argon. The mixture is stirred at 70° C. for 30 minutes, and 1 ml of a 2 M solution of sodium carbonate is then added. The reaction mixture is heated at 100° C. for 16 h. After cooling to RT, the mixture is filtered through silica gel. The solvent is removed under reduced pressure and the crude product is purified by preparative HPLC (YMC gel ODS-AQ S 5/15 μm; mobile phase A: water, mobile phase B: acetonitrile, gradient 0 min 30% B, 5 min 30% B, 50 min 95% B). This gives 87 mg (81% of theory).

LC-MS (method D): Rₜ=5.18 min

MS (ESIpos): m/z=520 (M+H)⁺

¹H-NMR (300 MHz, CDCl₃): δ=0.98 (d, 3H), 2.24 (s, 3H), 2.41 (m, 1H), 2.53 (s, 3H), 3.31 (dd, 1H), 4.15 (dd, 1H), 4.66 (s, 2H), 6.63 (d, 1H), 6.93 (s, 1H), 7.27 (m, 3H).

### Example 4

### [4-({3-Isopropyl-5-[4-(trifluoromethyl)phenyl]-2,3-dihydro-1H-pyrrolo[3,2-b]-pyridin-1-yl}sulphonyl)-2-methylphenoxy]acetic acid

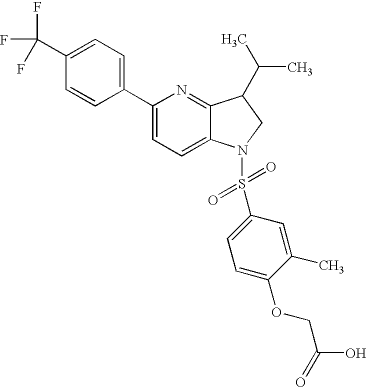 Step a):

### 5-Chloro-3-isopropyl-1H-pyrrolo[3,2-b]pyridine

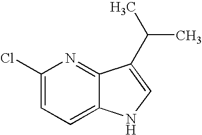

0.2 g (1.39 mmol) of 2-chloro-5-hydrazinopyridine (prepared according to GB-259 961 from 5-amino-2-chloropyridine) is dissolved in ethanol, and 0.16 g (1.8 mmol) of 3-methylbutanal is added. The mixture is stirred at RT for 30 minutes and the solvent is then removed under reduced pressure and the residue is dried under reduced pressure. 0.2 g (1.53 mmol) of anhydrous zinc chloride is then added to the intermediate and the mixture is heated in an oil bath at 170° C. After 30 minutes of stirring at this temperature, the mixture is cooled to RT. The crude product is taken up in dichloromethane and washed with dilute hydrochloric acid. After drying over magnesium sulphate, the solvent is removed under reduced pressure and the crude product is purified on silica gel (mobile phase: cyclohexane/ethyl acetate 1:1). This gives 133 mg (49% of theory).

LC-MS (method B): Rₜ=2.62 min

MS (ESIpos): m/z=195 (M+H)⁺

¹H-NMR (300 MHz, CDCl₃): δ=1.36 (d, 6H), 3.41 (m, 1H), 7.09 (d, 1H), 7.22 (s, 1H), 7.58 (d, 1H).

Step b):

### 3-Isopropyl-5-[4-(trifluoromethyl)phenyl]-1H-pyrrolo[3,2-b]pyridine

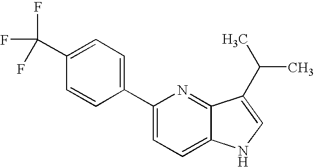

Under argon, 0.1 g (0.51 mmol) of 5-chloro-3-isopropyl-1H-pyrrolo[3,2-b]pyridine, 0.13 g (0.67 mmol) of 4-trifluoromethylphenylboronic acid and 0.018 g (0.026 mmol) of bis(triphenylphosphine)palladium(II) chloride are initially charged in 6 ml of DMF and heated at 70° C. for 30 minutes. After addition of 1 ml of a 2 M solution of sodium carbonate, the reaction mixture is heated at 100° C. overnight. After cooling, the mixture is filtered through silica gel. The solvent is removed under reduced pressure and the crude product is purified by preparative HPLC (YMC gel ODS-AQ S 5/15 μm; mobile phase A: water, mobile phase B: acetonitrile, gradient 0 min 30% B, 5 min 30% B, 50 min 95% B). This gives 100 mg (64% of theory).

LC-MS (method C): Rₜ=4.47 min

MS (ESIpos): m/z=305 (M+H)⁺

Step c):

### 3-Isopropyl-5-[4-(trifluoromethyl)phenyl]-2,3-dihydro-1H-pyrrolo[3,2-b]pyridine

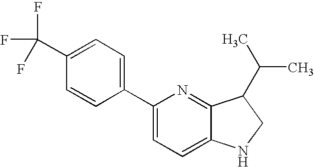

0.085 g (0.279 mmol) of 3-isopropyl-5-[4-(trifluoromethyl)phenyl]-1H-pyrrolo-[3,2-b]pyridine and 0.16 g (2.7 mmol) of Raney nickel are initially charged in 10 ml of decalin and hydrogenated at 80 bar and 180° C. for 16 h. The product is extracted with methanol and used without further purification for the next reaction step.

LC-MS (method D): Rₜ=5.00 min

MS (ESIpos): m/z=307 (M+H)⁺.

Step d):

### Ethyl [4-({3-isopropyl-5-[4-(trifluoromethyl)phenyl]-2,3-dihydro-1H-pyrrolo[3,2-b]-pyridin-1-yl}sulphonyl)-2-methylphenoxy]acetate

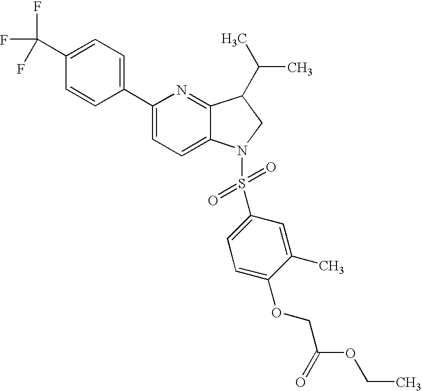

0.085 mg (0.277 mmol) of 3-isopropyl-5-[4-(trifluoromethyl)phenyl]-2,3-dihydro-1H-pyrrolo[3,2-b]pyridine are dissolved in 2 ml of absolute THF, and 0.081 g (0.277 mmol) of ethyl [4-(chlorosulphonyl)-2-methylphenoxy]acetate (Example 1/step e) and 0.085 ml (0.61 mmol) of triethylamine and 4 mg (0.028 mmol) of DMAP are added. The reaction mixture is warmed at 45° C. overnight. The mixture is then filtered and the solvent is removed under reduced pressure. The crude product is purified by preparative HPLC (YMC gel ODS-AQ S 5/15 μm; mobile phase A: water, mobile phase B: acetonitrile, gradient 0 min 30% B, 5 min 30% B, 50 min 95% B). This gives 37 mg (24% of theory).

LC-MS (method E): Rₜ=4.78 min

MS (ESIpos): m/z=563 (M+H)⁺

¹H-NMR (300 MHz, DMSO-d₆): δ=0.82 (d, 3H), 1.06 (d, 3H), 1.45 (m, 1H), 2.21 (m, 1H), 2.33 (s, 3H), 3.91 (m, 1H), 4.15 (m, 1H), 4.67 (s, 2H), 7.04 (d, 1H), 7.92 (m, 5H), 7.99 (d, 2H), 8.34 (d, 2H).

Step e):

### [4-({3-Isopropyl-5-[4-(trifluoromethyl)phenyl]-2,3-dihydro-1H-pyrrolo[3,2-b]-pyridin-1-yl}sulphonyl)-2-methylphenoxy]acetic acid

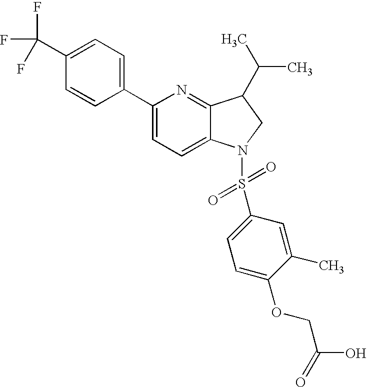

0.029 g (0.052 mmol) of ethyl [4-({3-isopropyl-5-[4-(trifluoromethyl)phenyl]-2,3-dihydro-1H-pyrrolo[3,2-b]pyridin-1-yl}sulphonyl)-2-methylphenoxy]acetate is dissolved in 1 ml of THF, and 0.5 ml of 1 N aqueous sodium hydroxide solution is added. The reaction mixture is stirred at RT overnight. The mixture is acidified with concentrated hydrochloric acid and then extracted with dichloromethane. The extract is dried over magnesium sulphate and the solvent is removed under reduced pressure. This gives 27 mg (97% of theory).

LC-MS (method E): Rₜ=4.43 min

MS (ESIpos): m/z=535 (M+H)⁺

¹H-NMR (300 MHz, DMSO-d₆): δ=0.82 (d, 3H), 1.06 (d, 3H), 1.45 (m, 1H), 2.21 (m, 1H), 2.33 (s, 3H), 3.91 (m, 1H), 4.15 (m, 1H), 4.67 (s, 2H), 7.04 (d, 1H), 7.92 (m, 5H), 7.99 (d, 2H), 8.34 (d, 2H).

### Example 5

### (4-{[5-(4-Trifluoromethylphenyl)-2,3-dihydro-3-spiro-1′-cyclohexyl-1H-indol-1-yl]-sulphonyl}-2-methylphenoxy)acetic acid

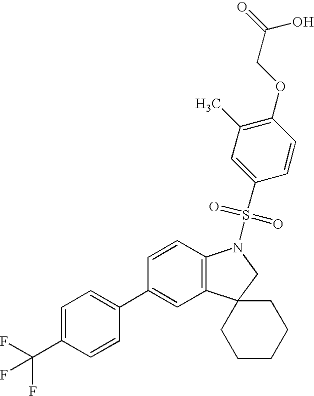 Step a):

### 4-Bromophenylhydrazine hydrochloride

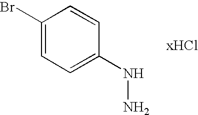

With stirring, a solution of 32.0 g (186 mmol) of 4-bromoaniline in 200 ml of concentrated hydrochloric acid is cooled to 0° C. At this temperature, a solution of 12.8 g (186 mmol) of sodium nitrite in 150 ml of water is added. The resulting diazonium solution is, with stirring at 0-4° C., added dropwise to a solution of 42.7 g (225 mmol) of tin(II) chloride in 100 ml of concentrated hydrochloric acid. The resulting precipitate is filtered off with suction and washed twice with in each case 50 ml of water and then recrystallized from isopropanol. This gives 17.2 g (41% of theory) of the product as a solid.

R_{f} (dichloromethane/methanol 40:1)=0.46

UV [nm]=198, 234, 284

MS (ESIpos): m/z=187, 189 [M+H]⁺

¹H-NMR (DMSO-d₆, 300 MHz): δ=6.93 (2H, d), 7.46 (2H, d), 8.39 (1H, s, br.), 10.23 (3H, s, br.).

Step b):

### 5-Bromo-2,3-dihydro-3-spiro-1′-cyclohexyl-1H-indole

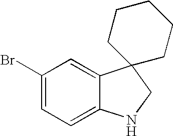

A mixture of 90 ml of toluene/acetonitrile (49:1) is flushed with argon for 5 minutes, and 6.00 g (26.8 mmol) of 4-bromophenylhydrazine hydrochloride are then added. 7.41 ml (96.2 mmol) of trifluoroacetic acid are then slowly added dropwise, while care is being taken that the temperature does not exceed 35° C. The temperature is then maintained at 35° C., and a solution of 3.27 g (29.2 mmol) of cyclohexanecarbaldehyde in 8.4 ml of toluene/acetonitrile (49:1) is then slowly added dropwise over a period of 2 h. The mixture is stirred at 35° C. for 4 h and at room temperature for 2 h. The mixture is then cooled to −10° C. and 8.0 ml of methanol are added. Over a period of 30 min, 1.64 mg (43.3 mmol) of solid sodium borohydride is added a little at a time; during the addition, the temperature must not exceed −2° C. After the addition has ended, the mixture is stirred at 0° C. for 1 h. 150 ml of a 6% strength by weight solution of ammonia in water are added and the phases are then separated and 3 ml each of acetonitrile and methanol are then added to the organic phase. The organic phase is then washed with 150 ml of a 15% strength solution of sodium chloride in water and dried over sodium sulphate. The organic phase is filtered through 150 g of silica gel and the filtercake is washed twice with in each case 200 ml of diethyl ether. The organic filtrate is concentrated under reduced pressure and chromatographed on 200 g of silica gel (70-230 mesh). First, the byproducts are eluted using cyclohexane, and the product is then eluted using a mixture of cyclohexane and diethyl ether (20:1). This gives 4.25 g (50% of theory) of a solid.

R_{f} (petroleum ether/ethyl acetate 5:1)=0.4

MS (ESIpos): m/z=266, 268 [M+H]⁺

UV [nm]=200, 270, 276

¹H-NMR (DMSO-d₆, 400 MHz): δ=1.20-1.69 (10H, m), 3.30 (2H, d), 5.65 (1H, s), 6.39 (1H, d), 7.01 (1H, dd), 7.07 (1H, d).

Step c):

### Ethyl {4-[(5-bromo-2,3-dihydro-3-spiro-1′-cyclohexyl-1H-indol-1-yl)sulphonyl]-2-methylphenoxy}acetate

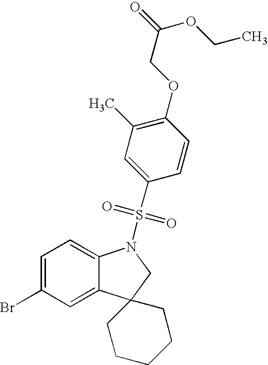

A solution of 4.5 g (16.9 mmol) of 5-bromo-2,3-dihydro-3-spiro-1′-cyclohexyl-1H-indole, 5.18 ml (37.2 mmol) of triethylamine and 210 mg (1.69 mmol) of 4-dimethylaminopyridine in 60 ml of absolute tetrahydrofuran is cooled to −5° C., and a solution of 4.95 g (16.91 mmol) of ethyl [4-(chlorosulphonyl)-2-methylphenoxy]-acetate (Example 1/step e) in 40 ml of abs. tetrahydrofuran is added dropwise at this temperature. The mixture is stirred at room temperature for 18 h, and 150 ml of distilled water are then added. The mixture is extracted three times with in each case 150 ml of ethyl acetate. The combined organic phases are washed with 200 ml of saturated sodium chloride solution, dried over sodium sulphate and concentrated under reduced pressure. The crude product is purified by flash chromatography using 150 g of silica gel (70-230 mesh). The mobile phase used is a mixture of cyclohexane and ethyl acetate (6:1). This gives 8.25 g (93% of theory) of the product as a solid foam.

R_{f} (petroleum ether/ethyl acetate 3:1)=0.6

MS (ESIpos): m/z=508, 510 [M+H]⁺

UV [nm]=202, 238, 258

¹H-NMR (DMSO-d₆, 300 MHz): δ=1.16 (3H, t), 1.05-1.55 (10H, m), 2.20 (3H, s), 3.67 (2H, s), 4.13 (2H, q), 4.89 (2H, s), 7.00 (1H, dd), 7.34-7.42 (3H, m), 7.55 (1H, dd), 7.68 (1H, d).

Step d):

### {4-[(5-Bromo-2,3-dihydro-3-spiro-1′-cyclohexyl-1H-indol-1-yl)sulphonyl]-2-methyl-phenoxy}acetic acid

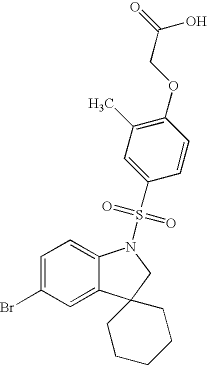

A solution of 0.53 g (9.47 mmol) of potassium hydroxide in 8 ml of water is added to a solution of 3.3 g (6.32 mmol) of ethyl {4-[(5-bromo-2,3-dihydro-3-spiro-1′-cyclohexyl-1H-indol-1-yl)sulphonyl]-2-methylphenoxy}acetate in 16 ml of tetrahydrofuran. The mixture is stirred at room temperature for one hour, and 0.49 g (3.16 mmol) of sodium dihydrogen phosphate dihydrate is then added. The tetrahydrofuran is removed under reduced pressure and the residue is diluted with 40 ml of water. The mixture is washed once with 40 ml of diethyl ether. The aqueous phase is adjusted to pH 2 using 1 N hydrochloric acid and extracted three times with in each case 40 ml of dichloromethane. The organic phase is dried over sodium sulphate and concentrated under reduced pressure. This gives 2.55 g (82% of theory) of the product as a solid foam.

R^{f} (petroleum ether/ethyl acetate 1:3)=0.14

MS (ESIpos): m/z=494, 496 [M+H]⁺

UV [nm]=206, 238, 258

¹H-NMR (DMSO-d₆, 200 MHz): δ=1.09-1.76 (10H, m), 2.19 (3H, s), 3.78 (2H, s), 4.78 (2H, s), 6.96 (1H, d), 7.37 (3H, d), 7.60 (1H, dd), 7.68 (1H, s), 13.2 (1H, s, br.).

Step e):

### (4-{[5-(4-Trifluoromethylphenyl)-2,3-dihydro-3-spiro-1′-cyclohexyl-1H-indol-1-yl]-sulphonyl}-2-methylphenoxy)acetic acid

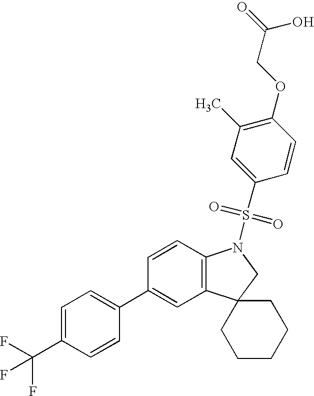

Under an atmosphere of argon, a solution of 170 mg (0.34 mmol) of {4-[(5-bromo-2,3-dihydro-3-spiro-1′-cyclohexyl-1H-indol-1-yl)sulphonyl]-2-methylphenoxy}acetic acid and 6.2 mg (8.5 μmol) of 1,1′-bis(diphenylphosphino)ferrocenepalladium(II) chloride in 3 ml of 1,2-dimethoxyethane is added to 84.9 mg (0.45 mmol) of 4-trifluoromethylboronic acid. With vigorous stirring, 0.76 ml of a 2 N solution of sodium carbonate are added. The mixture is stirred at 60° C. overnight. At room temperature, 8.50 mg (0.048 mmol) of 1,3,5-triazine-2,4,6-trithiol are added to the reaction solution. The pH is adjusted to 4-5 using 5 N trifluoroacetic acid in water and the solvent is then removed under reduced pressure. The residue is purified by RP-HPLC (Kroma-Sil 50×20 mm, mobile phase A: water with 0.3% trifluoroacetic acid, mobile phase B: acetonitrile, 0 min A:B=1:1, 7 min A:B=1:4, 8 min A:B=1:9). This gives 116 mg (61% of theory) of a solid.

R_{f} (methylene chloride/methanol 10:1)=0.28

MS (ESIpos): m/z=560 [M+H]⁺

UV [nm]=200, 292

¹H-NMR (DMSO-d₆, 200 MHz): δ=1.09-1.55 (10H, m), 2.20 (3H, s), 3.83 (2H, s), 4.79 (2H, s), 6.97 (1H, d), 7.57-7.88 (9H, m), 13.11 (1H, s).

### Example 6

### (4-{[5-(4-Methoxyphenyl)-2,3-dihydro-1H-indol-1-yl]sulphonyl}-2-methylphenoxy)-acetic acid

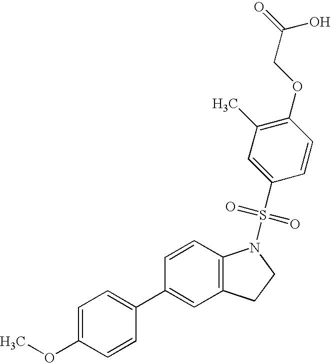 Step a):

### Ethyl {4-[(5-bromo-2,3-dihydro-1H-indol-1-yl)sulphonyl]-2-methylphenoxy}acetate

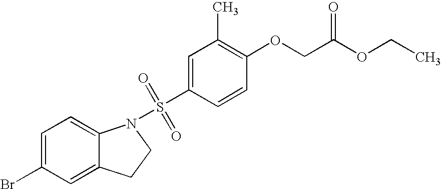

At a temperature of from −5 to 0° C., a solution of 1.17 g (4.00 mmol) of ethyl [4-(chlorosulphonyl)-2-methylphenoxy]acetate (Example 1/step e) in 8 ml of tetrahydrofuran is added dropwise to a solution of 792 mg (4.00 mmol) of 5-bromoindoline, 1.23 ml (8.80 mmol) of triethylamine and 48.9 mg (0.400 mmol) of 4-dimethylaminopyridine in 12 ml of tetrahydrofuran. The mixture is allowed to warm to room temperature and stirred for a further 2 h. 30 ml of water are added to the reaction solution, which is extracted three times with in each case 20 ml of ethyl acetate. The combined organic phases are dried with sodium sulphate and the solvent is removed under reduced pressure. This gives 1.5 g of crude product which is purified by flash chromatography (silica gel 70-230 mesh, mobile phase: cyclohexane/ethyl acetate 5:1). This gives 1.26 g (69% of theory) of the product as a solid.

R_{f} (petroleum ether/ethyl acetate 4:1)=0.25

MS (ESIpos): m/z=454 [M+H]⁺

UV [nm]=200, 208, 240

¹H-NMR (DMSO-d₆, 200 MHz): δ=1.17 (3H, t), 2.20 (3H, s), 2.93 (2H, t), 3.88 (2H, t), 4.14 (2H, q), 4.90 (2H, s), 7.00 (1H, d), 7.35-7.42 (3H m), 7.58-7.65 (2H, m).

Step b):

### 4-[(5-Bromo-2,3-dihydro-1H-indol-1-yl)sulphonyl]-2-methylphenoxyacetic acid

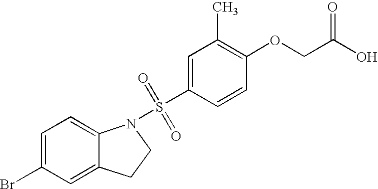

A solution of 57.4 mg (1.02 mmol) of potassium hydroxide in 1 ml of water is added to a solution of 310 mg (0.682 mmol) of ethyl {4-[(5-bromo-2,3-dihydro-1H-indol-1-yl)sulphonyl]-2-methylphenoxy}acetate in 2 ml of tetrahydrofuran. The mixture is stirred at room temperature for 45 minutes and the solvent is then removed under reduced pressure. The residue is diluted with 3 ml of water and adjusted to pH 2 using 1 N hydrochloric acid. The resulting precipitate is filtered off with suction through a filter cartridge. The precipitate is washed twice with in each case 2 ml of water and dried under reduced pressure. This gives 279 mg (96% of theory) of the product as a solid.

MS (ESIpos): m/z=426, 428 [M+H]⁺

UV [nm]=200, 238

¹H-NMR (DMSO-d₆, 300 MHz): δ=2.19 (3H, s), 2.93 (2H, t), 3.89 (2H, t), 4.79 (2H, s), 6.97 (1H, d), 7.31-7.41 (3H, m), 7.57-7.65 (2H, m).

Step c):

### (4-{[5-(4-Methoxyphenyl)-2,3-dihydro-1H-indol-1-yl]sulphonyl}-2-methylphenoxy)-acetic acid

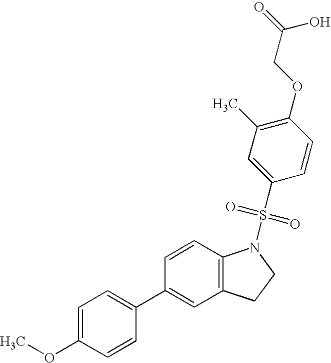

Under an atmosphere of argon, 54.7 mg (0.360 mmol) of 4-methoxyphenylboronic acid and 33.6 mg (0.792 mmol) of lithium chloride are initially charged. A solution of 128 mg (0.300 mmol) of 4-[(5-bromo-2,3-dihydro-1H-indol-1-yl)sulphonyl]-2-methylphenoxyacetic acid and 3.5 mg (3.0 μmol) of tetrakis(triphenylphosphine)-palladium(0) in 3 ml of 1,2-dimethoxyethane is added. With vigorous stirring, 660 μl of a 2 M solution of sodium carbonate in water are added. The mixture is heated at 60° C. overnight and then allowed to cool to room temperature. 8.50 mg (0.048 mmol) of 1,3,5-triazine-2,4,6-trithiol and 9.0 mg (0.041 mmol) of 2,2-bis(hydroxymethyl)-2,2′,2″-nitrilotriethanol are added to the reaction solution, and the mixture is concentrated under reduced pressure. The residue is washed with 2 ml of a solvent mixture of cyclohexane/ethyl acetate (2:1), taken up in a mixture of 3 ml of 1,2-dimethoxyethane and 0.6 ml of water and acidified with 0.66 ml of 5 N trifluoroacetic acid (pH≦4). The solvent is removed under reduced pressure and the residue is taken up in tetrahydrofuran and purified by preparative RP-HPLC (Kroma-Sil 50×20 mm, mobile phase A: water with 0.3% trifluoroacetic acid, mobile phase B: acetonitrile, 0 min A:B=9:1, 2 min A:B=9:1, 7 min A:B=1:9, 8 min A:B=1:9). This gives 107 mg (79% of theory) of the product as a lyophilisate.

MS (ESIpos): m/z=454 [M+H]⁺

UV [nm]=204, 246, 280

¹H-NMR (DMSO-d₆, 300 MHz): δ=2.19 (3H, s), 2.97 (2H, t), 3.77 (3H, s), 3.91 (2H, t), 4.78 (2H, s), 6.97 (3H, d), 7.39-7.53 (5H, m), 7.62-7.64 (2H, m).

### Example 7

### (4-{[5-(4-Trifluoromethylphenyl)-3,3-dimethyl-2,3-dihydro-1H-indol-1-yl]sulphonyl}-2-methylphenoxy)acetic acid

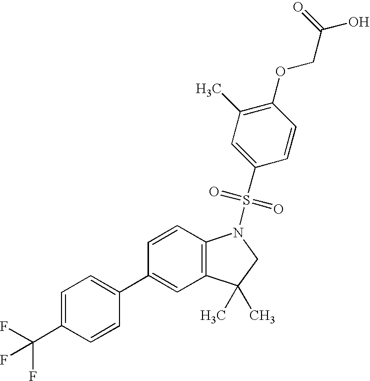 Step a):

### 5-Bromo-3,3-dimethylindoline

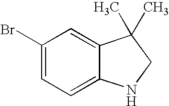

A mixture of 45 ml of toluene/acetonitrile (49:1) is flushed with argon for 5 minutes, and 3.00 g (13.4 mmol) of 4-bromophenylhydrazine are then added. 3.71 ml (48.1 mmol) of trifluoroacetic acid are then added slowly, while care is being taken that the temperature does not exceed 35° C. The temperature is then maintained at 35° C., and a solution of 1.05 g (14.6 mmol) of isobutyraldehyde in 4 ml of toluene/acetonitrile (49:1) is then slowly added dropwise over a period of 2 h. The mixture is stirred at 35° C. for 4 h and at room temperature for 2 h. The mixture is then cooled to −10° C., 4.0 ml of methanol are added and 819 mg (21.7 mmol) of solid sodium borohydride are then added a little at a time over a period of 30 min. Here, the temperature must not exceed −2° C. After the addition has ended, the mixture is stirred at 0° C. for 1 h. 150 ml of a 6% strength by weight solution of ammonia in water are added, the phases are then separated and 1.5 ml each of acetonitrile and methanol are added to the organic phase. The organic phase is then washed with 150 ml of a 15% strength solution of sodium chloride in water and dried over sodium sulphate. The mixture is filtered through 100 g of silica gel, and the filter cake is washed twice with in each case 200 ml of diethyl ether. The organic filtrate is concentrated under reduced pressure and chromatographed on 100 g of silica gel. Initially, the byproducts are eluted with cyclohexane, and the product is then eluted using a mixture of cyclohexane/diethyl ether (20:1). This gives 1.78 g (54% of theory) of the product as an oil.

R_{f} (petroleum ether/ethyl acetate 5:1)=0.47

UV [nm]=200, 268, 276

MS (ESIpos): m/z=226 [M+H]⁺

¹H-NMR (DMSO-d₆, 200 MHz): δ=1.20 (6H, s), 3.18 (2H, d), 5.66 (1H, s, br.), 6.42 (1H, d), 7.02 (1H, dd), 7.10 (1H, d).

Step b):

### Ethyl {4-[(5-bromo-3,3-dimethyl-2,3-dihydro-1H-indol-1-yl)sulphonyl]-2-methyl-phenoxy}acetate

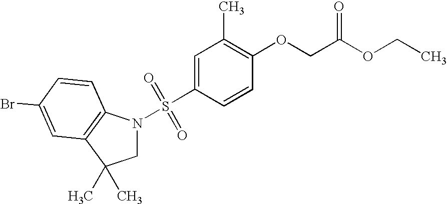

A solution of 920 mg (4.07 mmol) of 5-bromo-3,3-dimethylindoline, 906 mg (8.95 mmol) of triethylamine and 49.7 mg (0.407 mmol) of 4-dimethylaminopyridine in 12.5 ml of absolute tetrahydrofuran is cooled to −5° C., and a solution of 1.19 g (4.07 mmol) of ethyl [4-(chlorosulphonyl)-2-methylphenoxy]acetate (Example 1/step e) in 10 ml of abs. tetrahydrofuran is added dropwise at this temperature. The mixture is stirred at room temperature for 18 h, and 100 ml of distilled water are then added. The mixture is extracted three times with in each case 50 ml of ethyl acetate. The combined organic phases are washed with 200 ml of saturated sodium chloride solution, dried over sodium sulphate and concentrated under reduced pressure. The crude product is purified by flash chromatography using 150 g of silica gel. This gives 1.74 g (89% of theory) of the product as a solid foam.

R_{f} (petroleum ether/ethyl acetate 3:1)=0.48

### LC-MS (method A): R_{f}=5.18 min

MS (ESIpos): m/z=482 [M+H]⁺

UV [nm]=200, 238, 256

Step c):

### {4-[(5-Bromo-3,3-dimethyl-2,3-dihydro-1H-indol-1-yl)sulphonyl]-2-methylphenoxy}-acetic acid

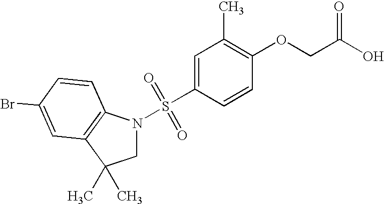

A solution of 173 mg (3.08 mmol) of potassium hydroxide and 2.5 ml of water is added to a solution of 990 mg (2.05 mmol) of ethyl {4-[(5-bromo-3,3-dimethyl-2,3-dihydro-1H-indol-1-yl)sulphonyl]-2-methylphenoxy}acetate in 5 ml of tetrahydrofuran, and the mixture is stirred at RT for 45 min. 160 mg (1.03 mmol) of sodium dihydrogen phosphate dihydrate are added. The solvent is removed under reduced pressure. 40 ml of water are added to the residue, and the mixture is washed with 20 ml of diethyl ether. The pH is then adjusted to 2 using a 1 N solution of hydrochloric acid, and the mixture is extracted three times with in each case 20 ml of dichloromethane. The organic phases are dried over sodium sulphate and the solvent is then removed under reduced pressure. This gives 805 mg (86% of theory) of the product as a solid foam.

R_{f} (dichloromethane/methanol 10:1)=0.31

MS (ESIpos): m/z=454, 456 [M+H]⁺

¹H-NMR (DMSO-d₆, 300 MHz): δ=1.10 (6H, s), 2.21 (3H, s), 3.64 (2H, s), 4.79 (2H, s), 6.99 (1H, d), 7.33-7.41 (3H, m), 7.62 (1H, dd), 7.65 (1H, s), 13.05 (1H, s, br.).

Step d):

### (4-{[5-(4-Trifluoromethylphenyl)-3,3-dimethyl-2,3-dihydro-1H-indol-1-yl]sulphonyl}-2-methylphenoxy)acetic acid

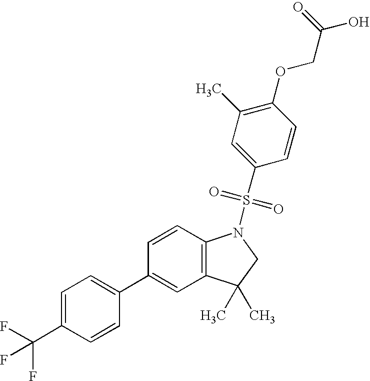

Under argon, a solution of 77.2 mg (0.17 mmol) of {4-[(5-bromo-3,3-dimethyl-2,3-dihydro-1H-indol-1-yl)sulphonyl]-2-methylphenoxy}acetic acid and 6.2 mg (8.5 μmol) of 1,1′-bis(diphenylphosphino)ferrocenepalladium(II) chloride in 1.5 ml of 1,2-dimethoxyethane is added to 38.0 g (0.20 mmol) of 4-trifluoromethylphenylboronic acid. With vigorous stirring, 374 μl of a 2 M solution of sodium carbonate in water are then added, and the mixture is stirred at 60° C. under argon for 17 h. To remove the palladium, 8.50 mg (0.048 mmol) of 1,3,5-triazine-2,4,6-trithiol are added to the reaction mixture, and the mixture is neutralized using 5 N trifluoroacetic acid in water. The mixture is concentrated under reduced pressure and the residue is taken up in 3 ml of a mixture of dichloromethane and methanol (5:1) and filtered through a cartridge filled with 2 g of silica gel. The product is eluted with 20 ml of the dichloromethane/methanol mixture (5:1) and the solvent is removed under reduced pressure. The residue is dissolved in a mixture of 400 μl of tetrahydrofuran and 200 μl of dimethyl sulphoxide and chromatographed by reversed-phase HPLC (Kroma-Sil, 50×20 mm, mobile phase A: water, mobile phase B: acetonitrile with 0.3% trifluoroacetic acid, gradient 0 min 50% A, 50% B; 7 min 20% A and 80% B; 8 min 10% A and 90% B). The solvent is removed under reduced pressure. This gives 46.1 mg (52% of theory) of the product as a solid.

LC-MS (method A): Rₜ=5.15 min

MS (ESIpos): m/z=520 [M+H]⁺

¹H-NMR (DMSO-d₆, 400 MHz): δ=1.19 (6H, s), 2.21 (3H, s), 3.70 (2H, s), 4.79 (2H, s), 6.99 (1H, d), 7.52-7.62 (3H, m), 7.67 (1H, d), 7.71 (1H, s), 7.76 (2H, d), 7.85 (2H, d).

The working examples 8-96 listed in the table below are obtained analogously to the processes described above:

| | | | LC- | | |
|---|---|---|---|---|---|
| | | | MS: | LC- | MW |
| Ex. | Synthesis | | R₁ | MS | found |
| No. | method | Structure | [min] | method | [M + H]⁺ |
| 8 | Analogous to Example 1 | 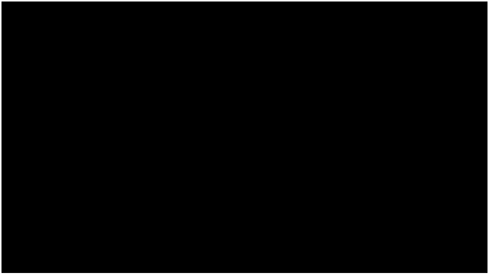 | 3.27 | B | 562 |
| 9 | Analogous to Example 1 | 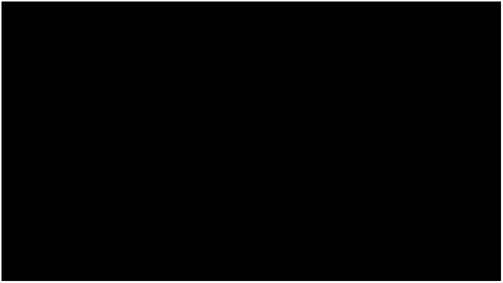 | 3.08 | B | 523 |
| 10 | Analogous to Example 1 | 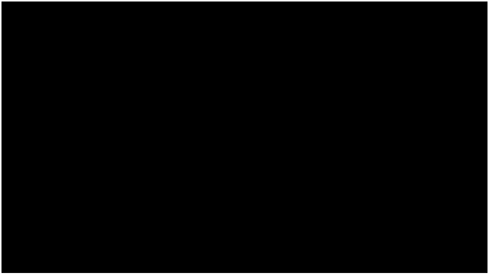 | 3.21 | B | 508 |
| 11 | Analogous to Example 1 | 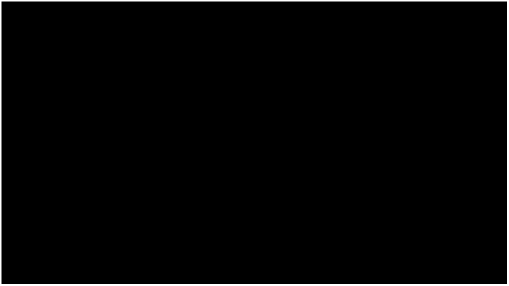 | 3.25 | B | 578 |
| 12 | Analogous to Example 1 | 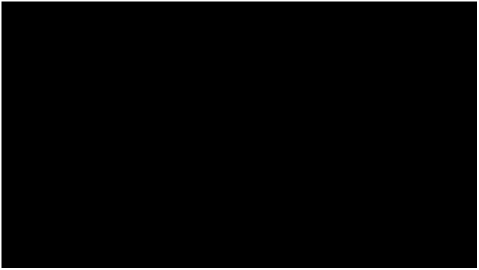 | 3.17 | B | 512 |
| 13 | Analogous to Example 1 | 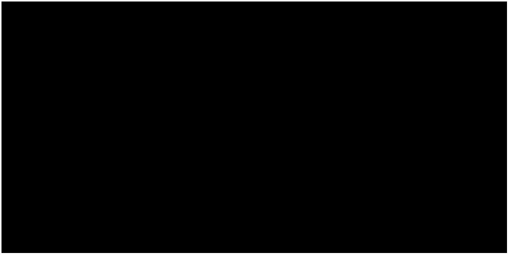 | 3.12 | B | 524 |
| 14 | Analogous to Example 1 | 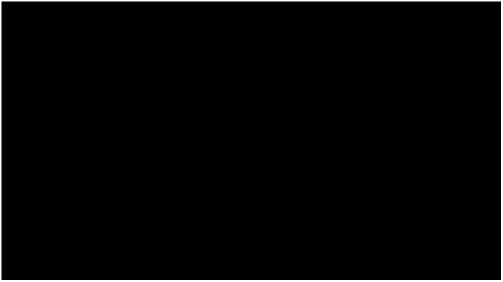 | 3.03 | B | 510 |
| 15 | Analogous to Example 1 | 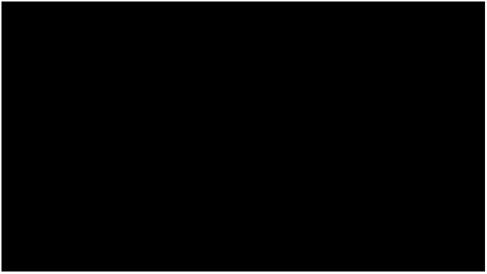 | 3.16 | B | 494 |
| 16 | Analogous to Example 1 | 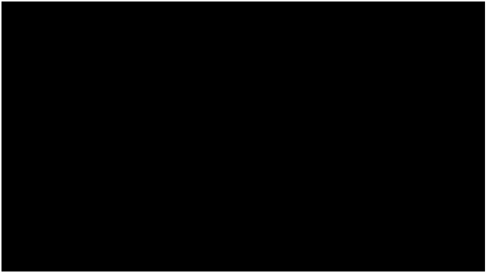 | 3.14 | B | 498 |
| 17 | Analogous to Example 1 | 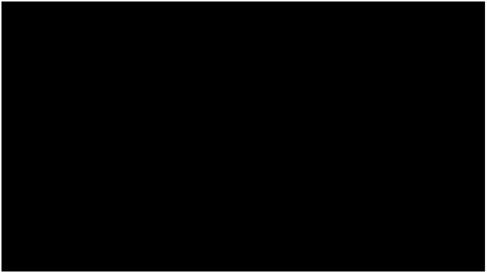 | 3.09 | B | 516 |
| 18 | Analogous to Example 5 | 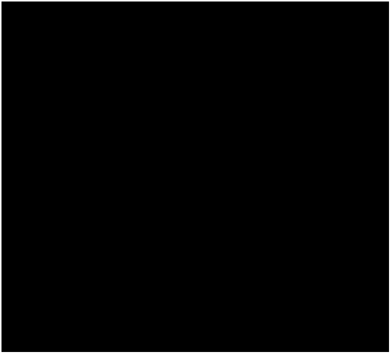 | 5.10 | D | 465 |
| 19 | Analogous to Example 5 | 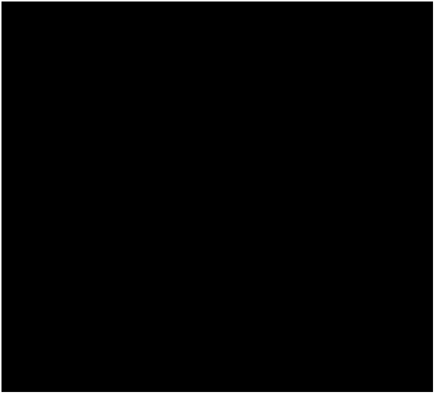 | 5.40 | D | 545 |
| 20 | Analogous to Example 5 | 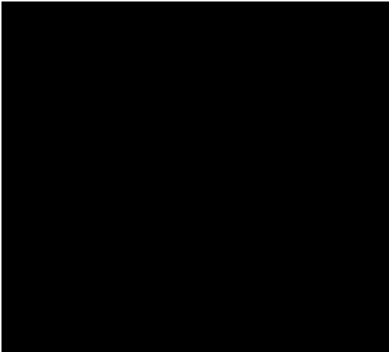 | 5.19 | D | 501 |
| 21 | Analogous to Example 5 | 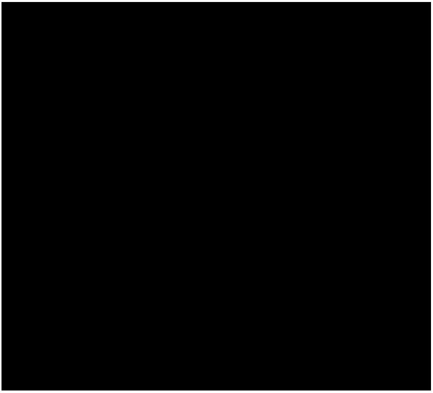 | 5.20 | D | 535 |
| 22 | Analogous to Example 5 | 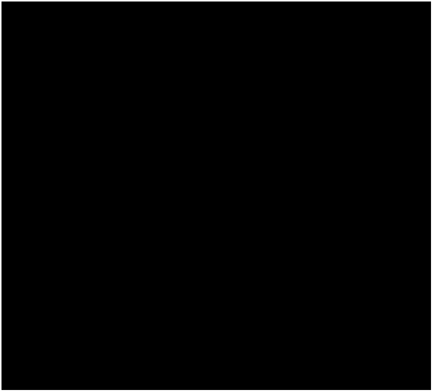 | 5.50 | D | 507 |
| 23 | Analogous to Example 5 | 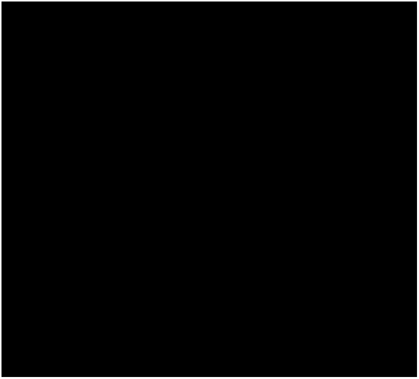 | 4.77 | D | 481 |
| 24 | Analogous to Example 5 | 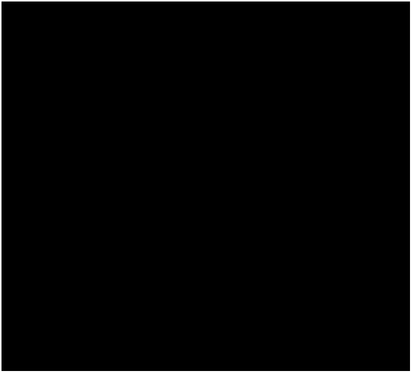 | 5.36 | D | 519 |
| 25 | Analogous to Example 5 | 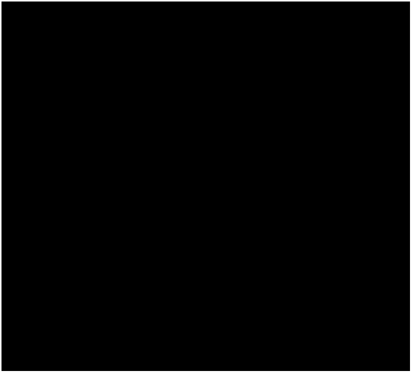 | 5.10 | D | 519 |
| 26 | Analogous to Example 5 | 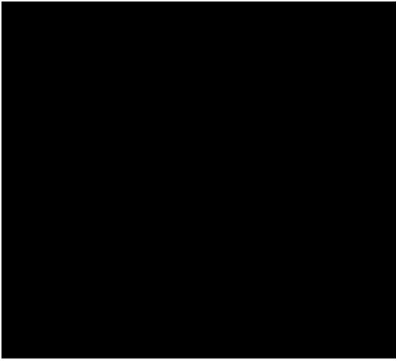 | 4.94 | D | 487 |
| 27 | Analogous to Example 5 | 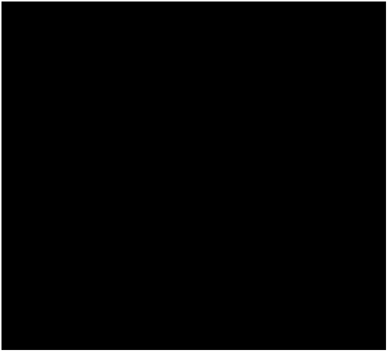 | 4.85 | D | 451 |
| 28 | Analogous to Example 5 | 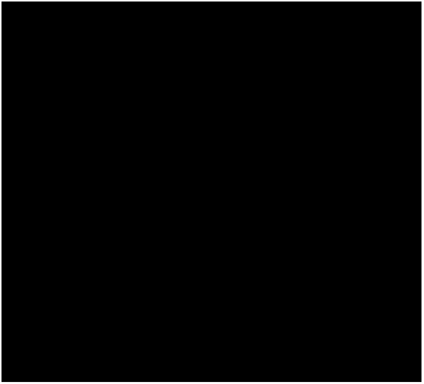 | 4.86 | D | 487 |
| 29 | Analogous to Example 5 | 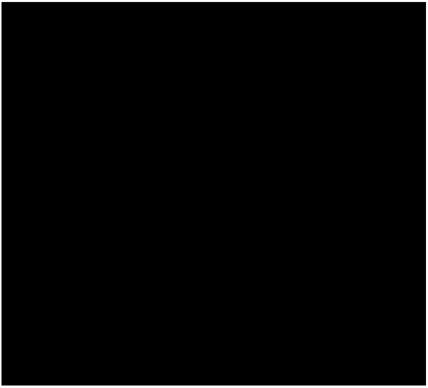 | 4.97 | D | 487 |
| 30 | Analogous to Example 5 | 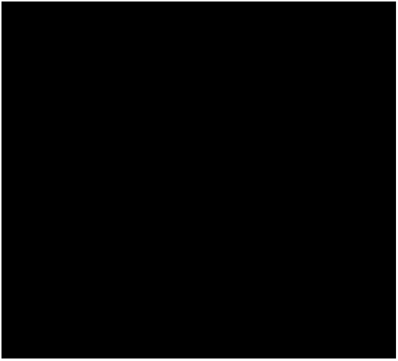 | 4.89 | D | 469 |
| 31 | Analogous to Example 5 | 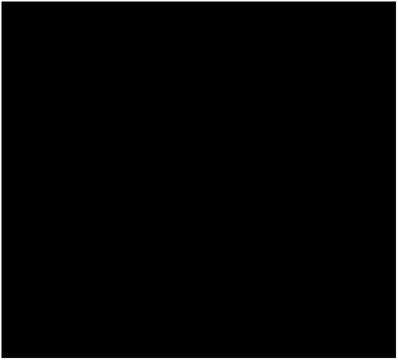 | 5.10 | D | 485 |
| 32 | Analogous to Example 5 | 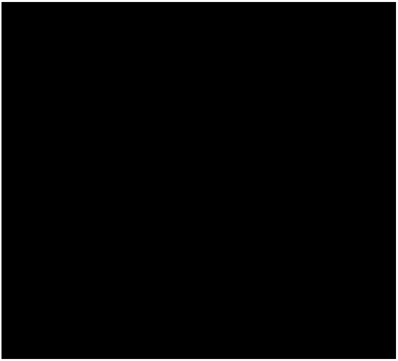 | 5.31 | D | 499 |
| 33 | Analogous to Example 5 | 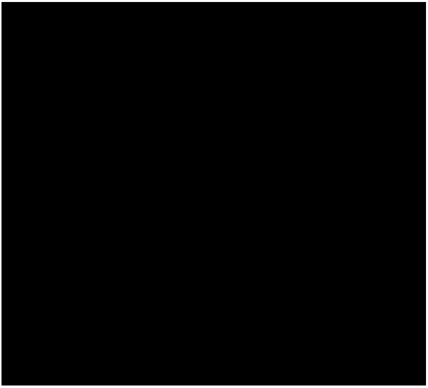 | 5.10 | D | 483 |
| 34 | Analogous to Example 5 | 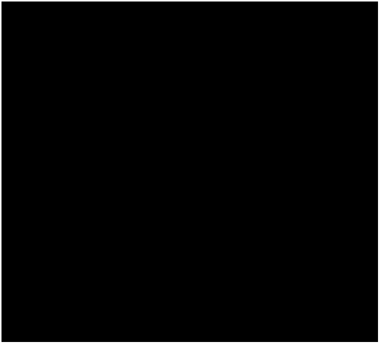 | 4.83 | D | 469 |
| 35 | Analogous to Example 5 | 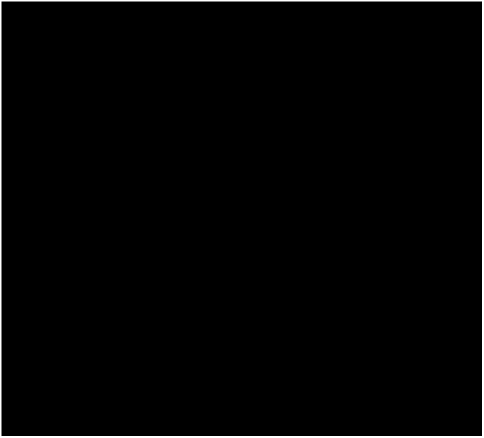 | 5.25 | D | 557 |
| 36 | Analogous to Example 5 | 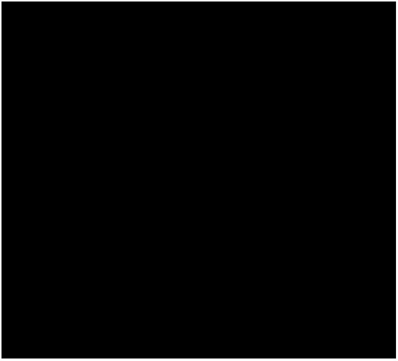 | 5.00 | D | 497 |
| 37 | Analogous to Example 5 | 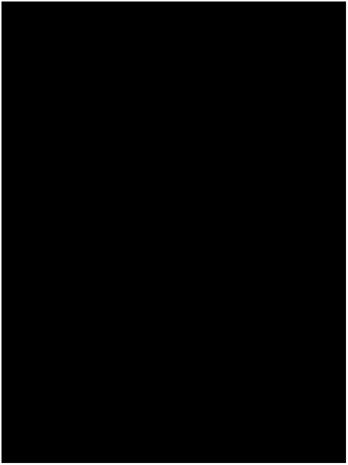 | 5.31 | D | 527 |
| 38 | Analogous to Example 5 | 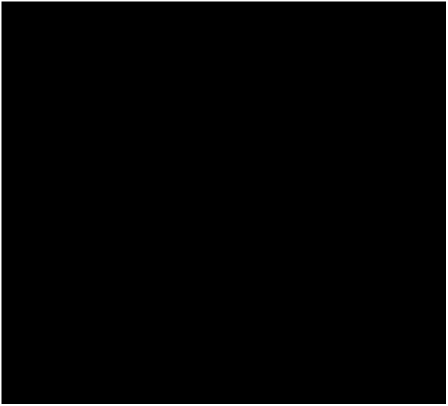 | 4.99 | D | 495 |
| 39 | Analogous to Example 5 | 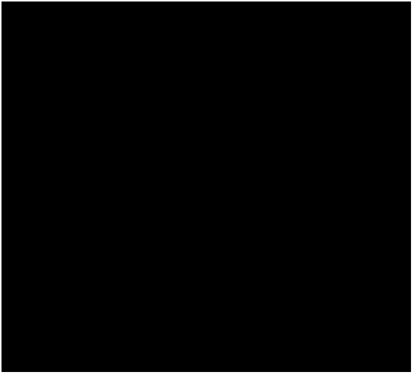 | 4.79 | D | 481 |
| 40 | Analogous to Example 5 | 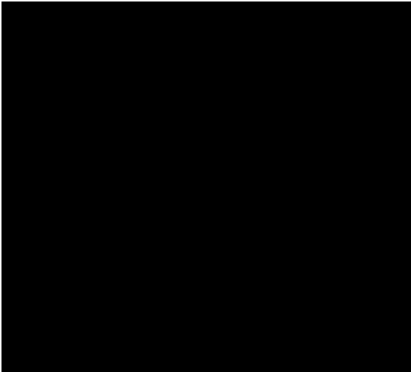 | 4.73 | D | 476 |
| 41 | Analogous to Example 5 | 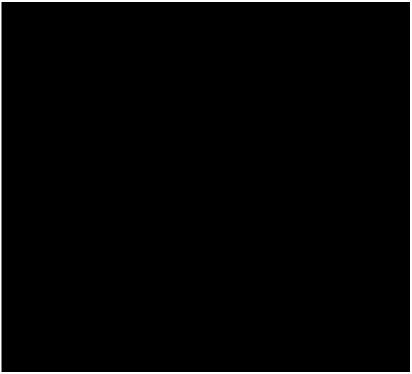 | 5.49 | C | 575 |
| 42 | Analogous to Example 5 | 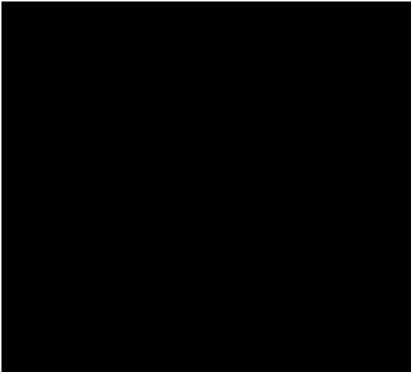 | 5.09 | C | 521 |
| 43 | Analogous to Example 5 | 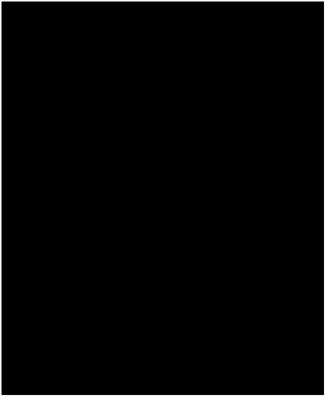 | 5.30 | C | 527 |
| 44 | Analogous to Example 5 | 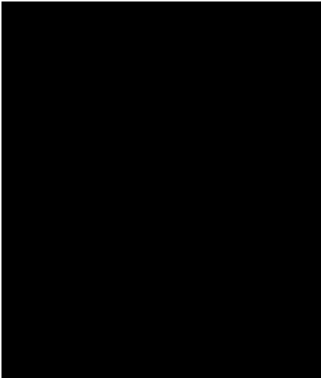 | 5.26 | D | 527 |
| 45 | Analogous to Example 5 | 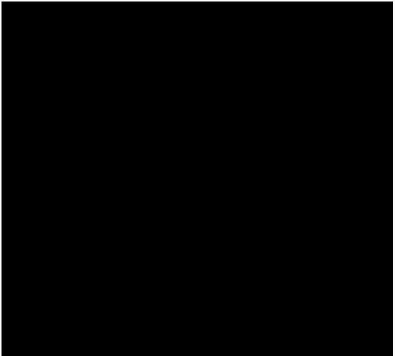 | 5.39 | C | 559 |
| 46 | Analogous to Example 5 | 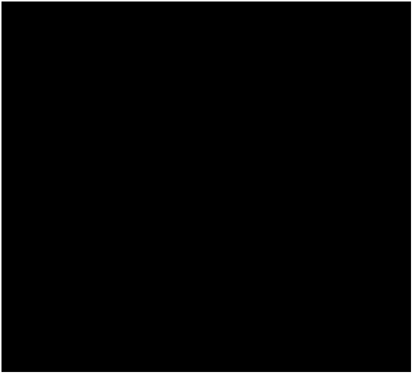 | 5.09 | C | 521 |
| 47 | Analogous to Example 5 | 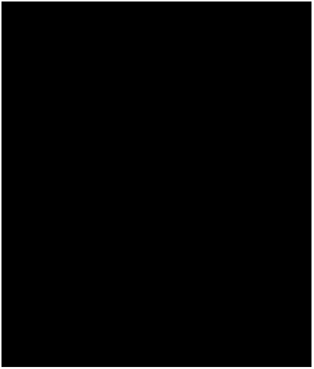 | 5.18 | C | 491 |
| 48 | Analogous to Example 5 | 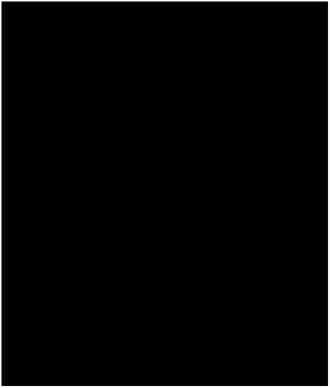 | 5.04 | C | 535 |
| 49 | Analogous to Example 5 | 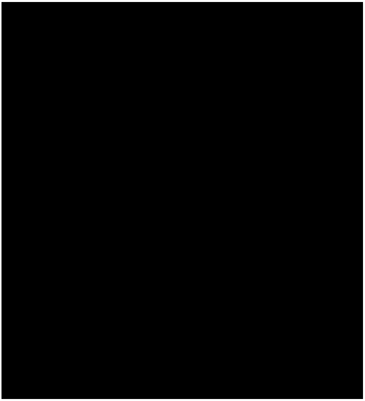 | 5.82 | C | 547 |
| 50 | Analogous to Example 5 | 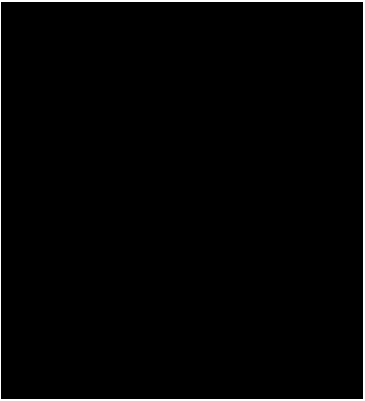 | 4.98 | D | 534 |
| 51 | Analogous to Example 5 | 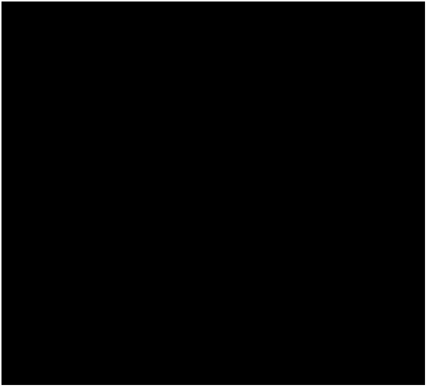 | 4.95 | C | 516 |
| 52 | Analogous to Example 5 | 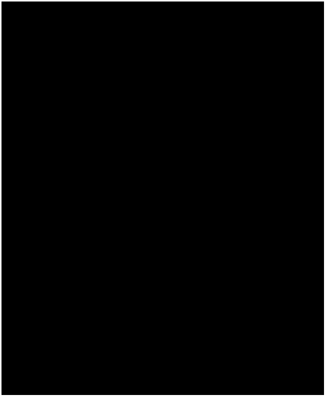 | 5.20 | C | 527 |
| 53 | Analogous to Example 5 | 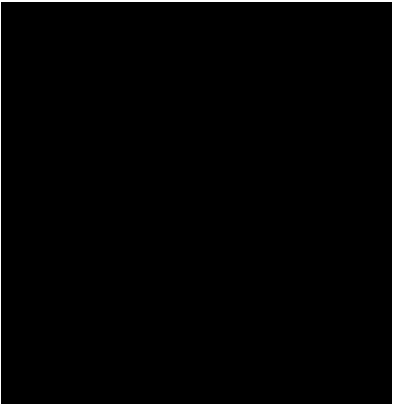 | 5.68 | C | 585 |
| 54 | Analogous to Example 5 | 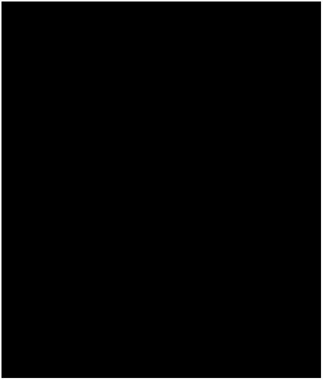 | 5.68 | C | 539 |
| 55 | Analogous to Example 5 | 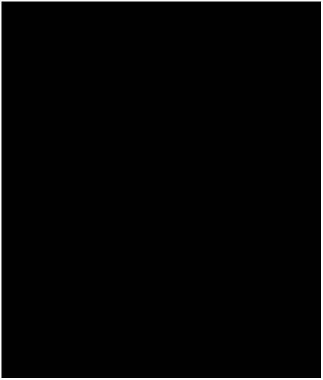 | 5.45 | C | 544 |
| 56 | Analogous to Example 5 | 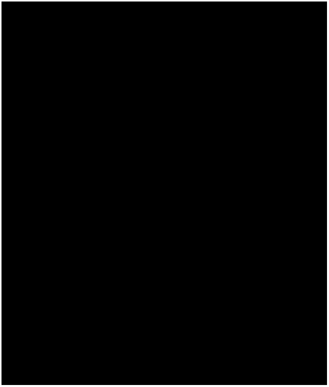 | 5.48 | C | 519 |
| 57 | Analogous to Example 5 | 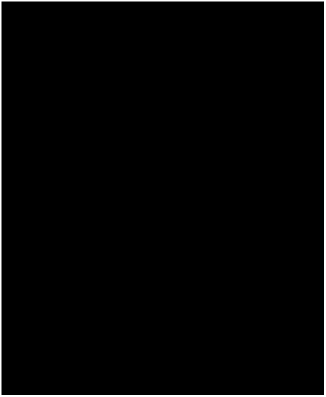 | 5.39 | D | 523 |
| 58 | Analogous to Example 5 | 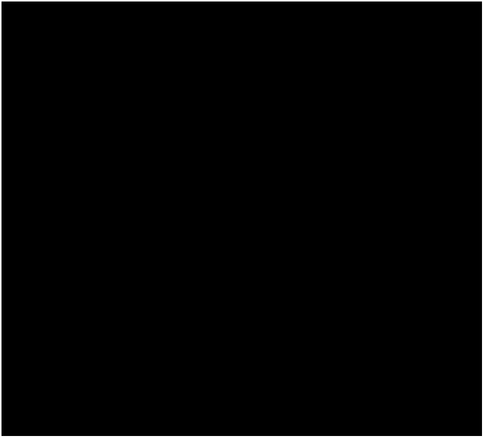 | 5.53 | D | 597 |
| 59 | Analogous to Example 5 | 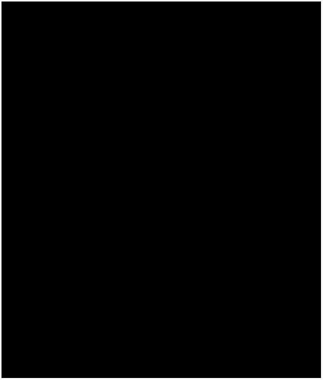 | 5.33 | F | 537 |
| 60 | Analogous to Example 5 | 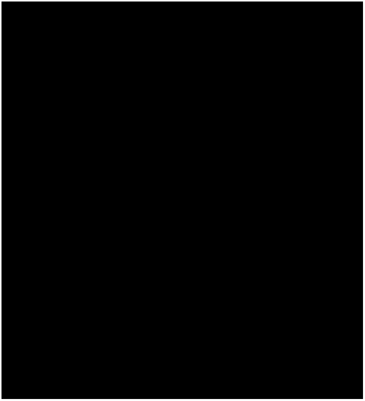 | 4.47 | F | 535 |
| 61 | Analogous to Example 5 | 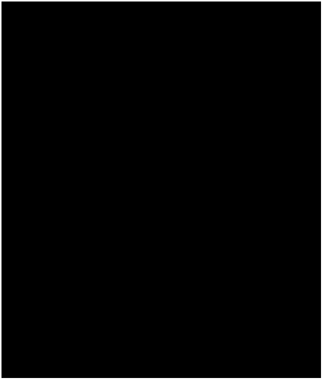 | 5.45 | C | 525 |
| 62 | Analogous to Example 5 | 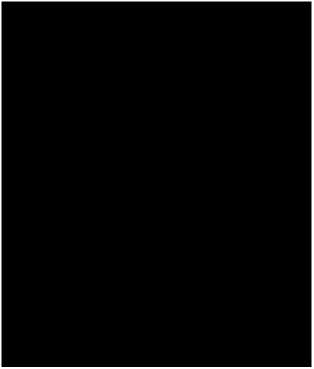 | 5.31 | C | 526 |
| 63 | Analogous to Example 5 | 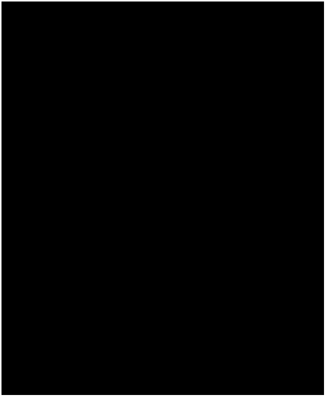 | 4.43 | F | 539 |
| 64 | Analogous to Example 5 | 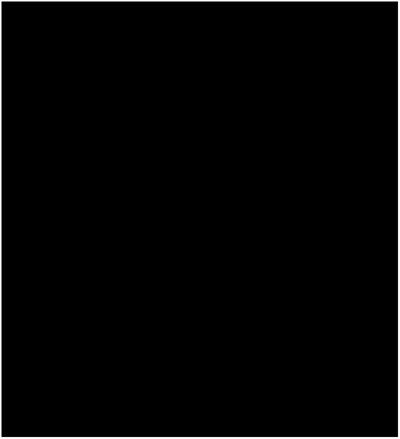 | 5.63 | C | 583 |
| 65 | Analogous to Example 5 | 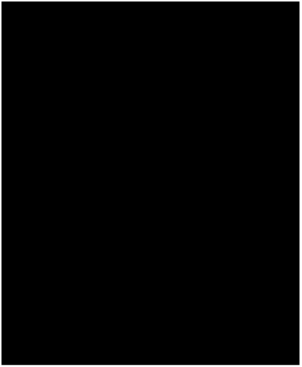 | 4.45 | F | 509 |
| 66 | Analogous to Example 1 | 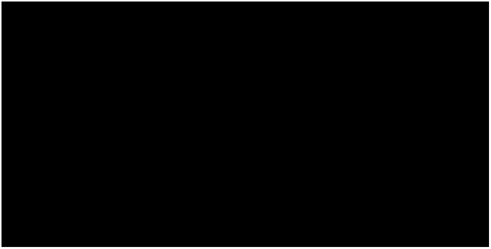 | 5.26 | E | 534 |
| 67 | Analogous to Example 1 | 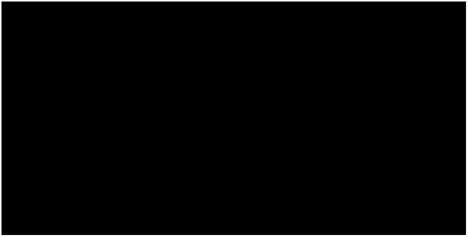 | 5.18 | E | 480 |
| 68 | Analogous to Example 1 | 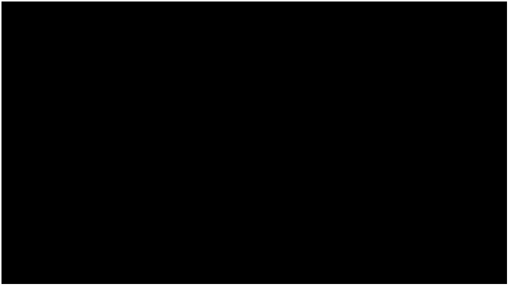 | 5.32 | E | 550 |
| 69 | Analogous to Example 1 | 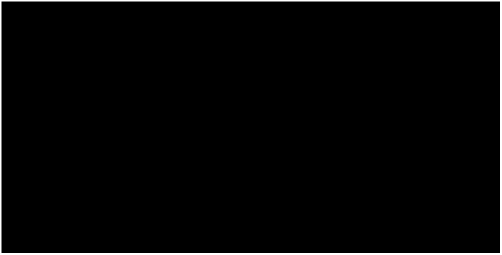 | 4.84 | E | 496 |
| 70 | Analogous to Example 1 | 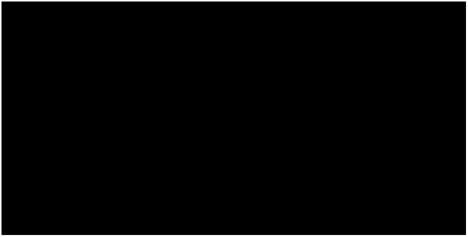 | 4.99 | E | 484 |
| 71 | Analogous to Example 1 | 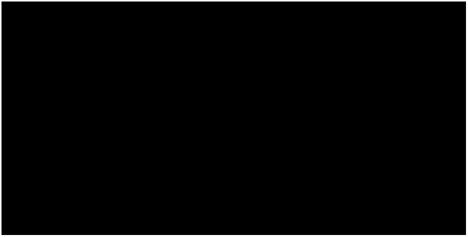 | 4.88 | E | 496 |
| 72 | Analogous to Example 1 | 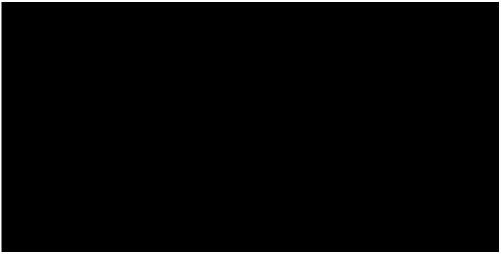 | 5.66 | E | 522 |
| 73 | Analogous to Example 1 | 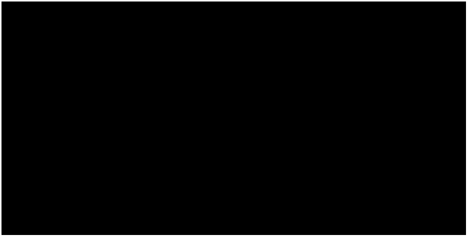 | 5.03 | E | 502 |
| 74 | Analogous to Example 5 | 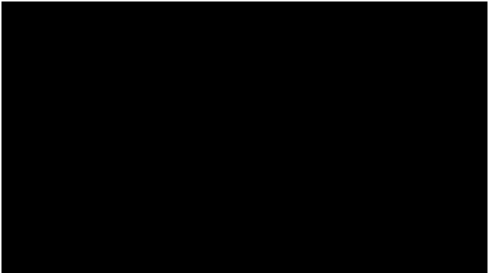 | 5.72 | E | 588 |
| 75 | Analogous to Example 5 | 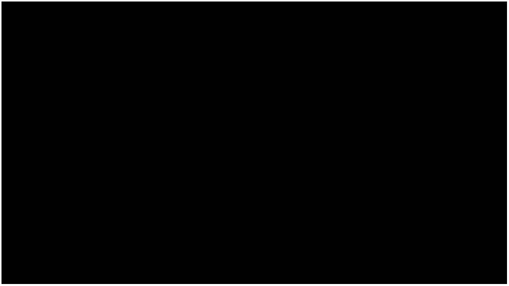 | 5.79 | E | 604 |
| 76 | Analogous to Example 5 | 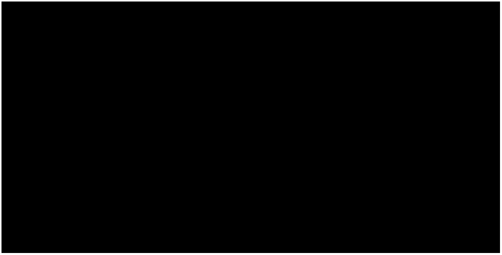 | 5.38 | E | 550 |
| 77 | Analogous to Example 5 | 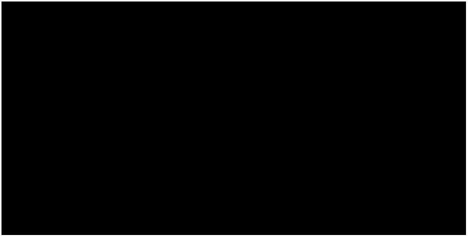 | 5.44 | E | 538 |
| 78 | Analogous to Example 5 | 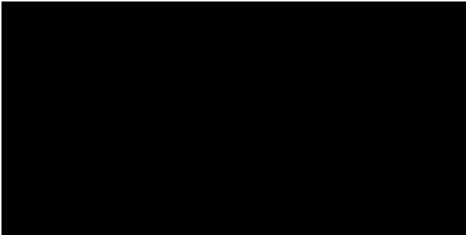 | 5.32 | E | 550 |
| 79 | Analogous to Example 5 | 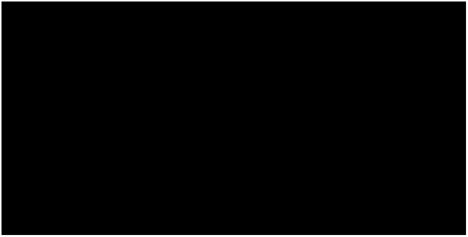 | 5.69 | E | 534 |
| 80 | Analogous to Example 5 | 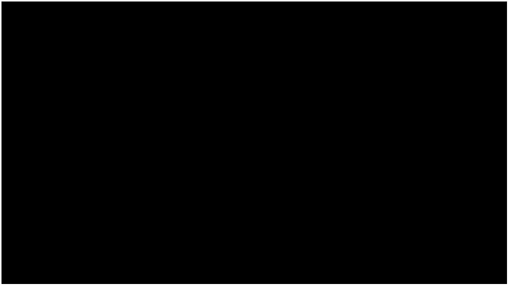 | 3.27 | B | 590 |
| 81 | Analogous to Example 5 | 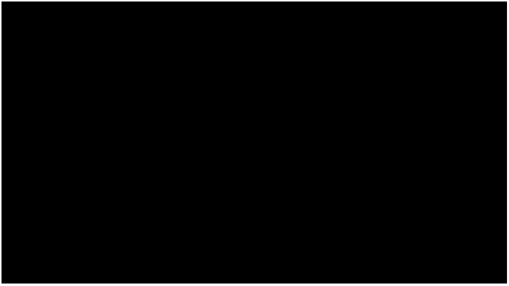 | 3.25 | B | 532 |
| 82 | Analogous to Example 5 | 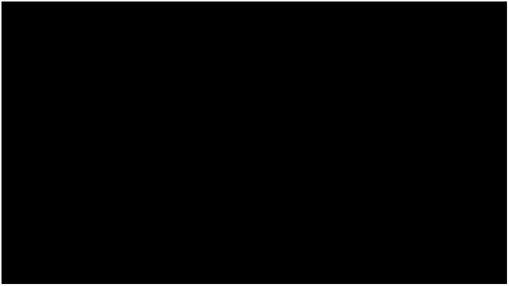 | 3.24 | B | 574 |
| 83 | Analogous to Example 5 | 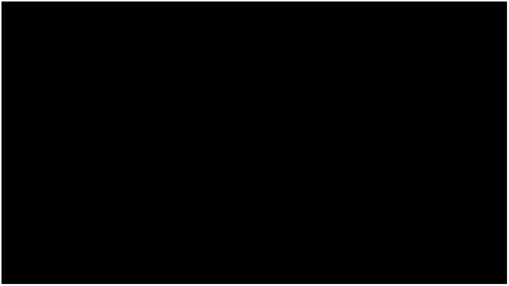 | 3.05 | B | 536 |
| 84 | Analogous to Example 5 | 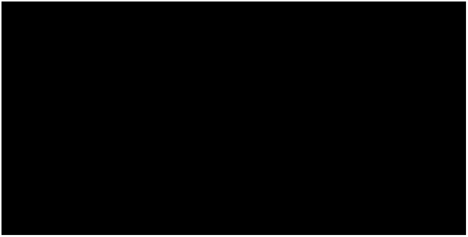 | 3.22 | B | 520 |
| 85 | Analogous to Example 5 | 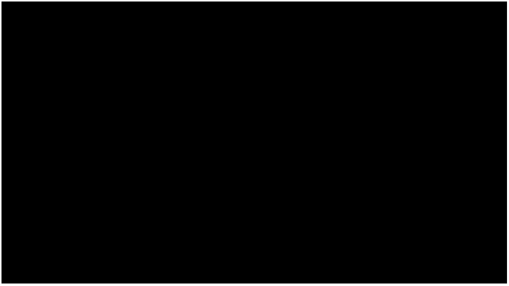 | 3.05 | B | 536 |
| 86 | Analogous to Example 1 | 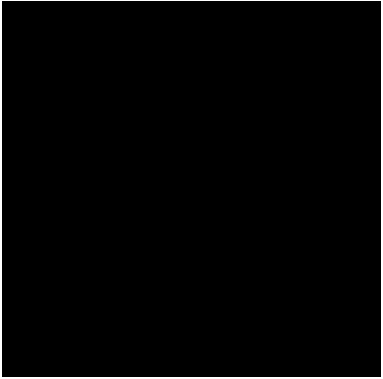 | 5.5 | E | 562 |
| 87 | Analogous to Example 1 | 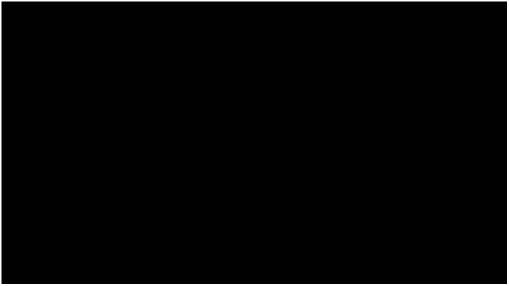 | 4.16 | E | 507 |
| 88 | Analogous to Example 1 | 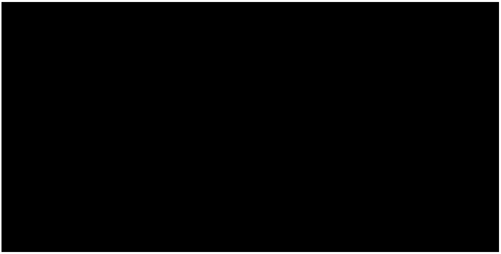 | 5.55 | D | 508 |
| 89 | Analogous to Example 1 | 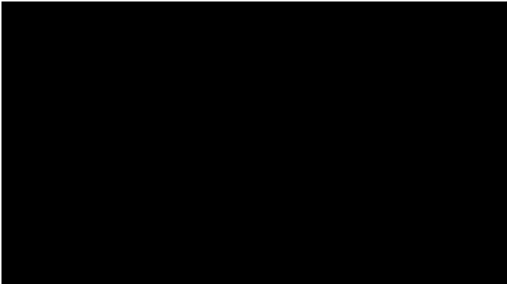 | 5.4 | E | 548 |
| 90 | Analogous to Example 1 | 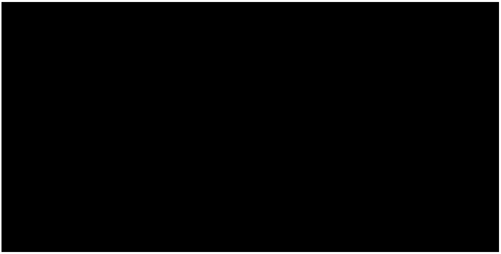 | 3.43 | B | 536 |
| 91 | Analogous to Example 1 | 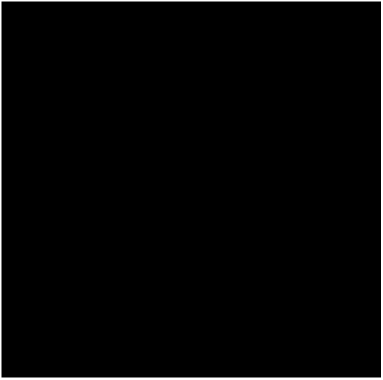 | 5.4 | E | 508 |
| 92 | Analogous to Example 1 | 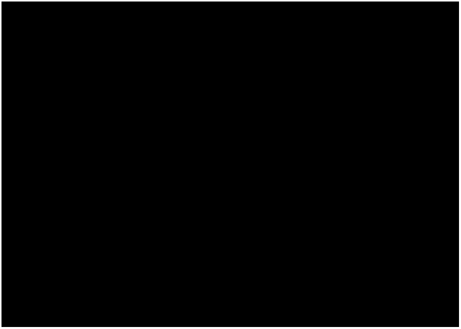 | 4.95 | C | 485 |
| 93 | Analogous to Example 1 | 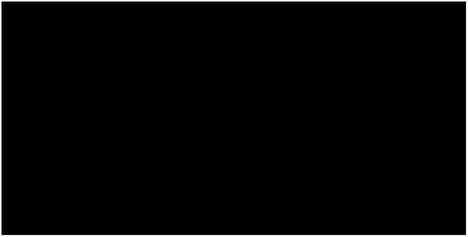 | 5.2 | C | 480 |
| 94 | Analogous to Example 1 | 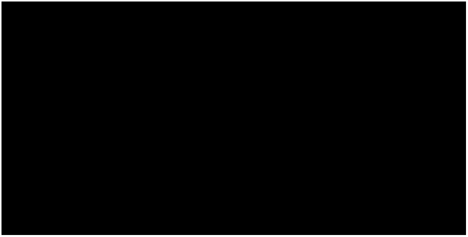 | 5.4 | E | 494 |
| 95 | Analogous to Example 1 | 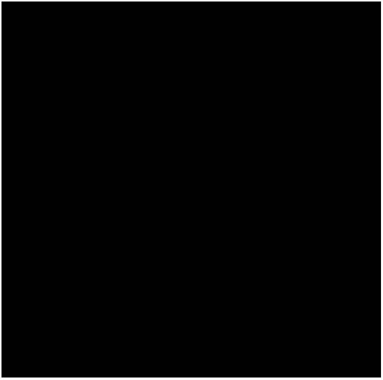 | 5.3 | E | 512 |
| 96 | Analogous to Example 1 | 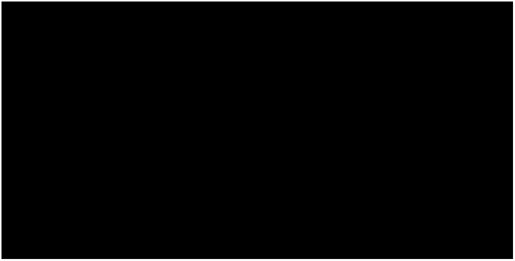 | 4.92 | C | 496 |

### Example A

Cellular Transactivation Assay:

Test Principle:

A cellular assay is used to identify activators of the peroxisome proliferator-activated receptor delta (PPAR-delta).

Since mammalian cells contain different endogenous nuclear receptors which may complicate an unambiguous interpretation of the results, an established chimera system is used in which the ligand binding domain of the human PPARδ receptor is fused to the DNA binding domain of the yeast transcription factor GAL4. The resulting GAL4-PPARδ chimera is co-transfected and stably expressed in CHO cells having a reporter construct.

Cloning:

The GAL4-PPARδ expression construct contains the ligand binding domain of PPARδ (amino acids 414-1326), which is PCR-amplified and cloned into the vector pcDNA3.1. This vector already contains the GAL4 DNA binding domain (amino acids 1-147) of the vector pFC2-dbd (Stratagene). The reporter construct, which contains five copies of the GAL4 binding site upstream of a thymidine kinase promoter, expresses firefly luciferase (Photinus pyralis) following activation and binding of GAL4-PPARδ.

Transactivation Assay (Luciferase Reporter):

CHO (chinese hamster ovary) cells are sown in CHO-A-SFM medium (GIBCO), supplemented by 2.5% foetal calf serum and 1% penicillin/streptomycin (GIBCO), at a cell density of 2×10³ cells per well in a 384-well plate (Greiner). The cells are cultivated at 37° C. for 48 h and then stimulated. To this end, the substances to be tested are taken up in the abovementioned medium and added to the cells. After a stimulation period of 24 hours, the luciferase activity is measured using a video camera. The relative light units measured give, as a function of the substance concentration, a sigmoidal stimulation curve. The EC₅₀ values are calculated using the computer program GraphPad PRISM (Version 3.02).

In this test, Working Examples 1-96 show EC₅₀ values in a range of from 1 to 200 nM.

### Example B

### Description of the Test for Finding Pharmacologically Active Substances which Increase HDL Cholesterol (HDL-C) Concentrations in the Serum of Transgenic Mice Transfected with the Human ApoA1 Gene (hApoA1) and/or Have an Effect on the Metabolic Syndrome of Adipose ob,ob Mice and Lower Their Blood Glucose Concentration:

The substances to be examined in vivo for their HDL-C-increasing activity are administered orally to male transgenic hApoA1 mice. One day prior to the start of the experiment, the animals are randomized into groups with the same number of animals, generally n=7-10. Throughout the experiment, the animals have drinking water and feed ad libitum. The substances are administered orally once a day for 7 days. To this end, the test substances are dissolved in a solution of Solutol HS 15+ethanol+saline (0.9%) in a ratio of 1+1+8 or in a solution of Solutol HS 15+saline (0.9%) in a ratio of 2+8. The dissolved substances are administered in a volume of 10 ml/kg of body weight using a stomach tube. Animals which have been treated in exactly the same manner but have only been given the solvent (10 ml/kg of body weight), without test substance, serve as control group.

Prior to the first administration of substance, a blood sample from each of the mice is taken by puncture of the retroorbital venous plexus, to determine ApoA1, serum cholesterol, HDL-C and serum triglycerides (TG) (zero value). Subsequently, using a stomach tube, the test substance is administered for the first time to the animals. 24 hours after the last administration of substance (i.e. on day 8 after the start of the treatment), another blood sample is taken from each animal by puncture of the retroorbital venous plexus, to determine the same parameters. The blood samples are centrifuged and, after the serum has been obtained, cholesterol and TG are determined photometrically using an EPOS Analyzer 5060 (Eppendorf-Gerätebau, Netheler & Hinz GmbH, Hamburg). The said determinations are carried out using commercial enzyme tests (Boehringer Mannheim, Mannheim).

To determine the HDL-C, the non-HDL-C fraction is precipitated using 20% PEG 8000 in 0.2 M glycine buffer pH 10. From the supernatant, the cholesterol is determined UV-photometrically (BIO-TEK Instruments, USA) in a 96-well plate using a commercial reagent (Ecoline 25, Merck, Darmstadt).

Human mouse-ApoA1 is determined with a Sandwich ELISA method using a polyclonal anti-human-ApoA1 antibody and a monoclonal anti-human-ApoA1 antibody (Biodesign International, USA). Quantification is carried out UV-photometrically (BIO-TEK Instruments, USA) using peroxidase-coupled anti-mouse-IGG antibodies (KPL, USA) and peroxidase substrate (KPL, USA)

The effect of the test substances on the HDL-C concentration is determined by subtracting the value measured for the 1st blood sample (zero value) from the value measured for the 2nd blood sample (after the treatment). The mean of the differences of all HDL-C values of one group is determined and compared to the mean of the differences of the control group.

Statistical evaluation is carried out using Student's t-test, after the variances have been checked for homogeneity.

Substances which increase the HDL-C of the treated animals in a statistically significant (p<0.05) manner by at least 15%, compared to that of the control group, are considered to be pharmacologically effective.

To examine substances for their effect on a metabolic syndrome, animals having an insulin resistance and increased blood glucose levels are used. To this end, C57B1/6J Lep <ob> mice are treated using the same protocol as for the transgenic ApoA1 mice. The serum lipids are determined as described above. In these animals, serum glucose is additionally determined, as a parameter for blood glucose. Serum glucose is determined enzymatically in an EPOS Analyzer 5060 (see above), using commercially available enzyme tests (Boehringer Mannheim).

A blood-glucose-lowering effect of the test substances is determined by subtracting the value measured for the 1st blood sample of an animal (zero value) from the value measured for the 2nd blood sample of the same animal (after the treatment). The mean of the differences of all serum glucose values of one group is determined and compared to the mean of the differences of the control group.

Statistical evaluation is carried out using Student's t-test, after the variances have been checked for homogeneity.

Substances which lower the serum glucose concentration of the treated animals in a statistically significant (p<0.05) manner by at least 10%, compared to the control group, are considered to be pharmacologically effective.

1. A compound of formula (I) 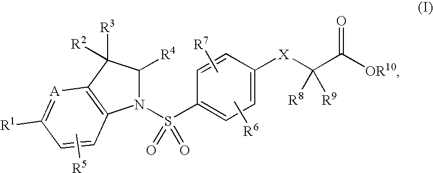
in which
A represents the group C—R¹¹ or represents N,
where
R¹¹ represents hydrogen or (C₁-C₄)-alkyl,
X represents O, S or CH₂,
R¹ represents (C₆-C₁₀)-aryl or represents 5- to 10-membered heteroaryl having up to three heteroatoms selected from the group consisting of N, O and S, which radicals may for their part each be mono- to trisubstituted by identical or different substituents selected from the group consisting of halogen, cyano, nitro, (C₁-C₆)-alkyl (which for its part may be substituted by hydroxyl), (C₁-C₆)-alkoxy, phenoxy, benzyloxy, trifluoromethyl, trifluoromethoxy, (C₂-C₆)-alkenyl, phenyl, benzyl, (C₁-C₆)-alkylthio, (C₁-C₆)-alkylsulphonyl, (C₁-C₆)-alkanoyl, (C₁-C₆)-alkoxycarbonyl, carboxyl, amino, (C₁-C₆)-acylamino, mono- and di-(C₁-C₆)-alkylamino and 5- or 6-membered heterocyclyl having up to two heteroatoms from the group consisting of N, O and S,
or represents a group of the formula 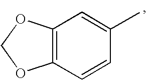
R² and R³ are identical or different and independently of one another represent hydrogen or (C₁-C₆)-alkyl or together with the carbon atom to which they are attached form a 3- to 7-membered spiro-linked cycloalkyl ring,
R⁴ represents hydrogen or (C₁-C₆)-alkyl,
R⁵ represents hydrogen or (C₁-C₆)-alkyl,
R⁶ represents hydrogen or (C₁-C₆)-alkyl,
R⁷ represents hydrogen, (C₁-C₆)-alkyl, (C₁-C₆)-alkoxy or halogen,
R⁸ and R⁹ are identical or different and independently of one another represent hydrogen or (C₁-C₄)-alkyl, and
R¹⁰ represents hydrogen or represents a hydrolysable group which can be degraded to the corresponding carboxylic acid,
or a pharmaceutically acceptable salt, solvate, or solvate of a salt thereof.

2. The compound of formula (I) according to claim 1, in which
A represents the group C—R¹¹ or represents N,
where
R¹¹ represents hydrogen or methyl,
X represents O or S,
R¹ represents phenyl or represents 5- or 6-membered heteroaryl having up to two heteroatoms selected from the group consisting of N, O and S, which radicals may for their part each be mono- or disubstituted by identical or different substituents selected from the group consisting of fluorine, chlorine, cyano, (C₁-C₄)-alkyl, (C₁-C₄)-alkoxy, phenoxy, benzyloxy, trifluoromethyl, trifluoromethoxy, vinyl, phenyl, benzyl, methylthio, methylsulphonyl, acetyl, propionyl, (C₁-C₄)-alkoxycarbonyl, amino, acetylamino, mono- and di-(C₁-C₄)-alkylamino,
R² and R³ are identical or different and independently of one another represent hydrogen or (C₁-C₄)-alkyl or together with the carbon atom to which they are attached form a 5- or 6-membered spiro-linked cycloalkyl ring,
R⁴ represents hydrogen or methyl,
R⁵ represents hydrogen, or methyl,
R⁶ represents hydrogen or methyl,
R⁷ represents hydrogen, (C₁-C₄)-alkyl, (C₁-C₄)-alkoxy, fluorine or chlorine,
R⁸ and R⁹ are identical or different and independently of one another represent hydrogen or methyl, and
R¹⁰ represents hydrogen.

3. The compound of formula (I) according to claim 1, in which
A represents CH or N,
X represents O,
R¹ represents phenyl or pyridyl which for their part may each be mono- or disubstituted by identical or different substituents selected from the group consisting of fluorine, chlorine, methyl, tert-butyl, methoxy, trifluoromethyl, trifluoromethoxy, methylthio, amino and dimethylamino,
R² represents hydrogen or methyl,
R³ represents methyl, isopropyl or tert-butyl, or
R² and R³ together with the carbon atom to which they are attached form a spiro-linked cyclohexane ring,
R⁴ represents hydrogen or methyl,
R⁵ represents hydrogen, methyl or ethyl,
R⁶ represents hydrogen or methyl,
R⁷ represents methyl,
R⁸ and R⁹ each represent hydrogen, and
R¹⁰ represents hydrogen.

4. A compound of the formula (I-A) 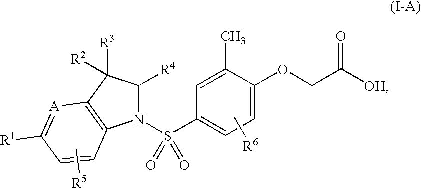
in which
R² represents hydrogen,
R³ represents methyl, isopropyl or tert-butyl, or
R² and R³ both represent methyl or together with the carbon atom to which they are attached form a spiro-linked cyclohexane ring, and
A, R¹, R⁴, R⁵ and R⁶ are each as defined in claim 1.

5. A process for preparing the compounds of formula (I) or (I-A) as defined in claims 1 and 4 respectively, characterized in that a compound of formula (II) 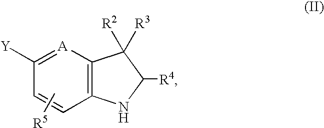
in which A, R², R³, R⁴ and R⁵ are each as defined in claim 1 and
Y represents chlorine or bromine,
is initially converted using a compound of formula (III) 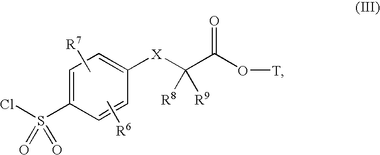
in which X, R⁶, R⁷, R⁸ and R⁹ are each as defined in claim 1 and
T represents benzyl or (C₁-C₆)-alkyl,
in an inert solvent in the presence of a base into a compound of formula (IV) 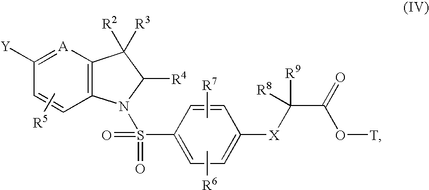
in which A, T, X, Y, R², R³, R⁴, R⁵, R⁶, R⁷, R⁸ and R⁹ are each as defined in claim 1,
this compound is then reacted in a coupling reaction with a compound of formula (V) 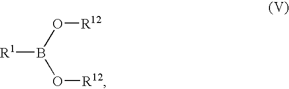
in which R¹ is as defined in claim 1 and
R¹² represents hydrogen or methyl or both radicals together form a —CH₂CH₂— or —C(CH₃)₂—C(CH₃)₂— bridge,
in an inert solvent in the presence of a suitable palladium catalyst and a base to give a compound of formula (I-B) 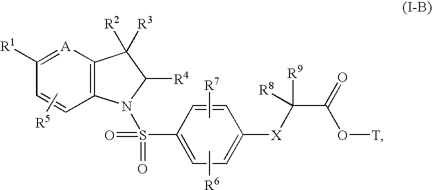
in which A, T, X, R¹, R², R³, R⁴, R⁵, R⁶, R⁷, R⁸ and R⁹ are each as defined in claim 1,
the compound (I-B) is then reacted with acid or base or, if T represents benzyl, also hydrogenolytically, to give the corresponding carboxylic acid of formula (I-C) 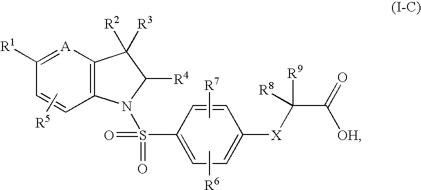
in which A, X, R¹, R², R³, R⁴, R⁵, R⁶, R⁷, R⁸ and R⁹ are each as defined in claim 1,
and the carboxylic acid (I-C) is, if appropriate, further modified by known esterification methods to give the compound of formula (I).

6. (cancelled)

7. A pharmaceutical composition, comprising at least one compound of the formula (I) or (I-A) as defined in claims 1 and 4, respectively, and an inert non-toxic pharmaceutically acceptable carrier, auxiliary, solvent, vehicle, emulsifier, or dispersant.

8. (cancelled)

9. (cancelled)

10. A method for the prevention and treatment of stroke, arteriosclerosis, coronary heart diseases and dyslipidaemias, for the prophylaxis of myocardial infarction and for the treatment of restenosis after coronary angioplasty or stenting comprising administering to a mammal an effective amount of a compound of formula (I) or formula (IV) as defined in claim 1 and 4, respectively.

11. (cancelled)
